(19) United States
(12) Patent Application Publication
(10) Pub. No.: US 2019/0359989 A1
(43) Pub. Date: Nov. 28, 2019

(54) FUSION PROTEIN AND APPLICATIONS THEREOF

(71) Applicants: CARSGEN THERAPEUTICS CO., LTD., Shanghai (CN); SHANGHAI CANCER INSTITUTE, Shanghai (CN)

(72) Inventors: Zonghai LI, Shanghai (CN); Xiuqi WU, Shanghai (CN); Huamao WANG, Shanghai (CN); Hua JIANG, Shanghai (CN); Bizhi SHI, Shanghai (CN)

(21) Appl. No.: 16/331,786

(22) PCT Filed: Jul. 6, 2017
(86) PCT No.: PCT/CN2017/092108

§ 371 Date: Mar. 8, 2019

(30) Foreign Application Priority Data
Sep. 9, 2016 (CN) 201610817555.4

(51) Int. Cl. C12N 15/62; C07K 14/725; C07K 14/705; C07K 14/71; C07K 14/82; C07K 16/28; C07K 16/30; C07K 16/32; C12N 15/85; C12N 5/0783; G01N 33/537; G01N 33/547; G01N 33/569; G01N 33/577

(52) U.S. Cl. CPC C12N 15/62; C07K 2319/02; C07K 14/70578; C07K 14/70596; C07K 14/71; C07K 14/82; C07K 16/2818; C07K 16/2863; C07K 16/2878; C07K 16/2887; C07K 16/30; C07K 16/32; C12N 15/85; C12N 5/0636; G01N 33/537; G01N 33/547; G01N 33/56972; G01N 33/577; C07K 14/7051

(57) ABSTRACT

Provided are a fusion protein comprising an antibody binding area and an endocytic functional area, the encoding nucleic acid of the protein, an expression vector of same, a host cell thereof, and an immune effector cell expressing the fusion protein or the endocytic functional area or further expressing a chimeric antigen receptors. Also provided are an immunoconjugate comprising a cell-killing part and an antibody conjugate in a specifically-binding immune effector cell or an antibody of the endocytic functional area, a reagent kit and uses of the immunoconjugate, and a method for specifically removing, selecting, or enriching and detecting the immune effector cell.

### TECHNICAL FIELD

The invention relates to the field of immunotherapy. In particular, the present invention relates to a fusion protein for controlling chimeric antigen receptor immune effector cells or TCR-T cells and uses thereof.

### BACKGROUND

In recent years, great progress has been achieved in adoptive cell therapy (ACT), such as CAR-T and TCR-T against malignant tumors, among which the development of CAR-T therapy is the most significant.

However, with the development of clinical trials of CAR-T cell therapy, there are many serious side effects, such as cytokine storms, off-target effects, etc. When serious adverse reactions occur, if the CAR-T cells are not inhibited in time, serious adverse, even life-threatening reactions will be incurred. Therefore, when using CAR-T treatment, it is necessary to introduce a safety switch at the same time, so that, when life-threatening reactions are incurred after CAR-T cells are used in a patient, the CAR-T cells in the body can be effectively and specifically cleared.

The safety switches currently used in cell therapy mainly include two forms: suicide genes and marker genes.

The suicide genes mainly include herpes simplex virus thymidine kinase (HSV-TK) and inducible cysteine-containing aspartate proteolytic enzyme 9 (inducible caspase-9, iCasp9). HSV-TK suicide gene greatly enhances the sensitivity of T cells to ganciclovir by expressing HSV-TK on T cells. However, since HSV-TK produces immunogenicity in patients, and patients receiving cell therapy will not be able to continue to use ganciclovir as an antiviral drug, both of which greatly limit the clinical use of HSV-TK. iCasp9 induces apoptosis of T cells expressing iCasp9 suicide gene by applying a small molecule drug (AP20187) in a patient. However, AP20187 has not been commercialized, thus limiting the popularity of iCasp9 suicide gene.

Marker genes expressing specific markers on the surface of T cells that can be recognized by antibodies, therefore T cells can be sorted, detected, and cleared. For example, it is reported in Hum Gene Ther, 11(4): 611-20 that the expression of CD20 receptor on the surface of T cells allows T cells to be recognized and killed by anti-CD20 monoclonal antibodies; and it is reported in Blood, 118(5): 1255-1263 that a truncated EGFR receptor capable of being recognized by an anti-EGFR monoclonal antibody was co-expressed on CAR-T cells.

The development of marker genes broadens the range of applications for safety switches, however, killing effects of marker genes depend on the complement system and activities of NK cells in vivo, since the killing effects are often mediated by complement dependent cytotoxicity (CDC) and antibody-dependent cell-mediated cytotoxicity (ADCC). When the complement system or NK cell activities in a patient's body is defective, killing effects of marker genes are often limited. These shortcomings limit the application of these marker genes.

Therefore, with the rapid development of cell therapy and clinical application, there is an urgent need in the art for a technical means capable of effectively and specifically killing T cells.

### SUMMARY OF THE INVENTION

The object of the present invention is to provide an immune effector cell expressing a chimeric antigen receptor, wherein the surface of the immune effector cell simultaneously expresses a fusion protein, by which the immune effector cell can be highly effectively killed by a specific antibody-drug conjugate.

In a first aspect, an immune effector cell which expresses a chimeric antigen receptor on its surface is provided in the present invention, the immune cell further expressing a fusion protein of formula I,

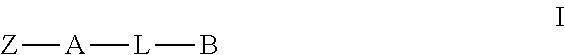

Wherein Z is an optional signal peptide;

A is an antibody binding region;

L is an optional linker moiety; and

B is an endocytic domain.

The present invention also provides an immune effector cell expressing a chimeric antigen receptor, wherein the immune cell further expresses a fusion protein comprising an antibody binding region and an endocytic domain.

In a preferred embodiment, the antibody binding region is a polypeptide that is absent in normal cells, or is in a concealed state in normal cells, or is low expressed in normal cells.

In a specific embodiment, the antibody binding region is selected from the following antigens or fragments thereof: EGFRvIII, EGFR, CD20, CD22, CD19, BCMA, proBDNF precursor protein, GPC3, CLD18.2, CLD6, mesothelin, PD-L1, PD-1, WT-1, IL13Ra2, Her-2, Her-1, Her-3;

Preferably, the antibody binding region comprises any one of the following amino acid sequences or comprises an amino acid sequence having at least 90%, 91%, 92%, 93%, 94%, 95%, 96%, 97%, 98% or 99% identity with the following amino acid sequence: SEQ ID NO: 28, 29, 33, 34, 35, 36, 37, 38, 39, 40, 41, 42, 43;

More preferably, the antibody binding region comprises an active fragment of any one of the following amino acid sequences: SEQ ID NO: 28, 29, 33, 34, 35, 36, 37, 38, 39, 40, 41, 42, 43.

In a specific embodiment, the antibody binding region specifically binds to an EGFR antibody.

In a preferred embodiment, the extracellular portion of the chimeric antigen receptor does not have binding ability to the fusion protein.

In a specific embodiment, the endocytic domain is derived from a folate receptor, LDL, CD30, CD33, CD3, EGFR, TFR1; preferably derived from a folate receptor and CD30; more preferably, the endocytic domain has an amino acid sequence of SEQ ID NO: 32 or 44, or an amino acid sequence having at least 90%, 91%, 92%, 93%, 94%, 95%, 96%, 97%, 98% or 99% identity with SEQ ID NO: 32 or 44, or is an active fragment of an amino acid sequence of SEQ ID NO: 32 or 44.

In a specific embodiment, the signal peptide is a folate receptor signal peptide.

In a specific embodiment, the fusion protein has an amino acid sequence of SEQ ID NO: 10 or comprises an amino acid sequence having at least 90%, 91%, 92%, 93%, 94%, 95%, 96%, 97%, 98% or 99% identity with SEQ ID NO: 10, or an active fragment thereof.

In a specific embodiment, the fusion protein and the chimeric antigen receptor are separately expressed or fusion-expressed on the surface of the immune effector cell, preferably separately expressed.

In a preferred embodiment, the endocytic domain is capable of transferring a substance binding to the antibody binding region or endocytic domain into the immune effector cell.

In a preferred embodiment, after transferred into the immune effector cell, the substance initiates killing of the immune effector cell.

In a preferred embodiment, the substance is an antibody-drug conjugate (ADC).

In a second aspect, an immune effector cell expressing a chimeric antigen receptor is provided in the present invention, the cell further expresses an endocytic domain, and the endocytic domain is capable of transferring a substance binding to the endocytic domain into the immune effector cell.

In a preferred embodiment, after transferred into the immune effector cell, the substance initiates killing of the immune effector cells.

In a preferred embodiment, the substance is an antibody drug conjugate (ADC).

In a specific embodiment, the endocytic domain is derived from a folate receptor, LDL, CD30, CD33, CD3, EGFR, TFR1; preferably derived from a folate receptor and CD30; more preferably, the endocytic domain having an amino acid sequence of SEQ ID NO: 32 or 44, or an amino acid sequence having at least 90%, 91%, 92%, 93%, 94%, 95%, 96%, 97%, 98% or 99% identity with SEQ ID NO: 32 or 44, or an active fragment of an amino acid sequence of SEQ ID NO: 32 or 44.

In a specific embodiment, the endocytic domain and the chimeric antigen receptor are separately expressed or fusion-expressed on the surface of the immune effector cell, preferably separately expressed.

In a third aspect, a fusion protein of Formula I is provided in the present invention,

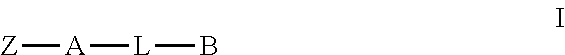

Wherein Z is an optional signal peptide;

A is an antibody binding region;

L is an optional linker moiety; and

B is an endocytic domain.

The invention also provides a fusion protein comprising an antibody binding region and an endocytic domain.

In a preferred embodiment, the antibody binding region is a polypeptide that is absent in normal cells, or is in a concealed state in normal cells, or is low expressed in normal cells.

In a specific embodiment, the antibody binding region is selected from the following antigens or fragments thereof: EGFRvIII, EGFR, CD20, CD22, CD19, BCMA, proBDNF precursor protein, GPC3, CLD18.2, CLD6, mesothelin, PD-L1, PD-1, WT-1, IL13Ra2, Her-2, Her-1, Her-3;

Preferably, the antibody binding region comprises any one of the following amino acid sequences or comprises an amino acid sequence having at least 90%, 91%, 92%, 93%, 94%, 95%, 96%, 97%, 98% or 99% identity with the following amino acid sequence: SEQ ID NO: 28, 29, 33, 34, 35, 36, 37, 38, 39, 40, 41, 42, 43;

More preferably, the antibody binding region comprises an active fragment of any one of the following amino acid sequences: SEQ ID NO: 28, 29, 33, 34, 35, 36, 37, 38, 39, 40, 41, 42, 43.

In a specific embodiment, the antibody binding region specifically binds to an EGFR antibody.

In a specific embodiment, the endocytic domain is derived from a folate receptor, LDL, CD30, CD33, CD3, EGFR, TFR1; preferably derived from a folate receptor and CD30; more preferably, the endocytic domain has an amino acid sequence of SEQ ID NO: 32 or 44, or an amino acid sequence having at least 90%, 91%, 92%, 93%, 94%, 95%, 96%, 97%, 98% or 99% identity with SEQ ID NO: 32 or 44, or is an active fragment of an amino acid sequence of SEQ ID NO: 32 or 44.

In a specific embodiment, the signal peptide is a folate receptor signal peptide.

In a specific embodiment, the fusion protein has an amino acid sequence of SEQ ID NO: 10 or comprises an amino acid sequence having at least 90%, 91%, 92%, 93%, 94%, 95%, 96%, 97%, 98% or 99% identity with SEQ ID NO: 10, or an active fragment thereof.

In a fourth aspect, the encoding nucleic acid of the fusion protein of the third aspect of the invention is provided in the present invention.

In a fifth aspect, an expression vector comprising the encoding nucleic acid of the fourth aspect of the invention is provided in the present invention.

In a sixth aspect, a host cell is provided in the present invention, comprising the expression vector of the fifth aspect of the present invention or having the encoding nucleic acid of the fourth aspect of the present invention integrated into its genome.

In a seventh aspect, an immunoconjugate is provided in the present invention comprising:

A cell-killing functional moiety; and

An antibody that specifically binds to the antibody binding region or endocytic domain in the immune effector cell of the first aspect of the present invention, or an antibody that specifically binds to the endocytic domain in an immune effector cell of the second aspect of the present invention.

In a preferred embodiment, the cell-killing functional moiety is a small molecule drug or a killing cytokine, including but not limited to MMAF, Auristatin, calicheamicin, maytansine, maytansine, doxorubicin, paclitaxel, 5-fluorouracil, methotrexate, DM1, DM4, MGBA, SN-38 (see: Sassoon I, Blanc V. Antibody-Drug Conjugate (ADC) Clinical Pipeline: A Review[M]//Antibody-Drug Conjugates. Humana Press, 2013: 1-27).

In an eighth aspect, the use of the immunoconjugate of the seventh aspect of the present invention for specifically killing the immune effector cells of the first or second aspect of the present invention is provided in the present invention.

In a ninth aspect, a kit is provided in the present invention, comprising the immune effector cell of the first or second aspect of the present invention or the immunoconjugate of the seventh aspect of the present invention.

In a tenth aspect, a method for specifically eliminating the immune effector cells of the first or second aspect of the present invention is provided in the present invention, comprising the step of administering the immunoconjugate of the seventh aspect of the invention.

In a preferred embodiment, the immunoconjugate is administered at a concentration of not less than 0.1 μg/ml; preferably from 0.1 μg/ml to 100 μg/ml; more preferably, from 1 μg/ml to 100 μg/ml; and more preferably, 10 μg/ml.

In a preferred embodiment, the substance exhibits substantially non-killing effects against cells not expressing the fusion protein of the third aspect of the present invention.

In an eleventh aspect, a method for sorting or enriching the immune effector cells of the first or second aspect of the present invention is provided in the present invention, comprising the steps of:

Adding a sorting reagent to the system comprising the immune effector cell, wherein the sorting reagent comprises a substance capable of specifically binding to the antibody binding region or endocytic domain in the immune effector cell of the first aspect of the present invention, or a substance capable of specifically binding to the endocytic domain in the immune effector cell according to the second aspect of the present invention; and

A step of separating the substance binding to the immune effector cells from the system.

In a preferred embodiment, the substance is an antibody or an active fragment thereof.

In a specific embodiment, the substance capable of specifically binding to the antibody binding region or endocytic domain in the immune effector cell of the first aspect of the present invention, or the substance capable of specifically binding to the endocytic domain in the immune effector cell according to the second aspect of the present invention is immobilized on a solid phase carrier, thereby separating the substance binding to the immune effector cells from the system.

In a preferred embodiment, the solid support is a magnetic bead or a resin.

In a preferred embodiment, the substance is an antibody or an active fragment thereof.

In a preferred embodiment, the concentration of the sorting reagent is not less than 0.01 μg/ml; preferably 0.01 μg/ml˜100 μg/ml; more preferably, 0.1 μg/ml˜10 μg/ml; and more preferably, 10 μg/ml.

In a preferred embodiment, the sorting reagent exhibits a sorting efficiency of greater than 80% for the immune effector cells.

In a twelfth aspect, a method for detecting an immune effector cell of the first or second aspect of the present invention is provided in the present invention, the method comprising:

Administering a detection reagent that specifically binds to an antibody binding region or endocytic domain in the immune effector cell of the first aspect of the present invention or a detection reagent that specifically binds to the endocytic domain in the immune effector cell of the second aspect of the present invention, wherein the detection reagent is linked to a detectable label; and

Detecting a complex formed by the detection reagent and the immune effector cell.

In a preferred embodiment, the detection reagent is an antibody or an active fragment thereof.

It is to be understood that the above various technical features of the present invention and the various technical features specifically described hereinafter (as in the embodiments) may be combined with each other within the scope of the present invention, to form a new or preferred technical solution, which will not be repeated one by one due to the limited length of the specification.

### DESCRIPTION OF FIGURES

FIG. 1 shows a schematic diagram of the construction of a fusion protein of the present invention;

FIG. 2A shows a Flow CytoMetry pattern of T cells expressing FR806 fusion protein and CH12 antibodies; and FIG. 2B shows a Flow CytoMetry pattern of Keratinocyte expressing EGFR and HEK-293T cells as well as CH12 antibody;

FIG. 3 shows the affinity of CH12-biotin for FR806;

FIG. 4 shows results of sorting FR806 positive cells using CH12-biotin;

FIG. 5 shows the endocytosis of CH12 antibody mediated by FR806 fusion receptor;

FIG. 6A shows the binding ability of CH12-MMAF and CH12 to FR806-expressing T cells; FIG. 6B shows the endocytosis of CH12-MMAF by FR806+ T-cells; FIG. 6C shows killing effects of different concentrations of CH12-MMAF at different times on T cells expressing FR806; and FIG. 6D shows killing effects of CH12-MMAF on human Keratinocy cells;

FIG. 7A shows the killing effects of CH12-MMAF and free MMAF detected by CCK8 on FR806 positive and negative T cells; and FIG. 7B shows the killing effects of CH12-MMAF and free MMAF on FR806 positive and negative 293T cells;

FIG. 8A shows the linking pattern of FR806 with αCD19CAR and eGFP; FIG. 8B shows results of flow analysis of CAR-T cells with CAR19 and FR806 expressed on their surface; and FIG. 8C shows sorting T cells with FR806-CAR19 using CH12-biotin;

FIG. 9A shows the linking manner of FR806 and αCD19CAR, and FIG. 9B shows results of flow cytometry of T cells expressing CAR19 and FR806;

FIG. 10A shows killing results on tumor cells by CAR-T cells expressing FR806 and not expressing FR806; and FIG. 10B shows results of cytokine release of CAR-T cells expressing FR806 and not expressing FR806;

FIG. 11A shows killing effects of CH12-MMAF on T cells co-expressing FR806 and CAR; and FIG. 11B shows killing effects of CH12-MMAF concentrations on T cells co-expressing FR806 and CAR;

FIG. 12A is a graph showing eGFP positive rate of human CD3+ cells by gating analysis;

FIG. 12B shows in vivo killing effects of CH12-MMAF and physiological saline on FR806-CAR19-eGFP-expressing CAR-T cells; FIG. 12C shows the detection rate of CD3+/eGFP+ in mouse blood, spleen, and bone marrow, after administration of CH12-MMAF and saline, n=6; and FIG. 12B shows results of flow analysis of CAR-T cells with CAR19 and FR806 expressed on the surface;

FIG. 13 shows killing effects of CH12-MMAF on T cells co-expressing CD30806 and CAR.

### MODES FOR CARRYING OUT THE INVENTION

Through extensive and intensive research, the inventors have unexpectedly discovered that a fusion protein comprising an antibody binding region, an optional linker moiety and an endocytic domain can be expressed on an immune effector cell expressing a chimeric antigen receptor, and the resulting immune effector cell can be killed by a specific antibody to the antibody binding region. The antibody binding region is preferably absent from normal cells, and when an antibody specifically binding to the antibody binding region is administered, the antibody won't bind to normal cells, and therefore does not kill normal cells; and even if the antibody binding region is exposed on normal cells, too much impacts won't be caused on normal cells since the amount of cells used to kill immune cells is small. Moreover, since the fusion protein is capable of mediating endocytosis, the killing effects on cells are completed inside the cell membrane, and the killing ability is remarkable. An immune effector cell expressing a chimeric antigen receptor which only expressing an endocytic domain is also provided in the present invention, and the endocytic domain is capable of transferring a substance binding to the endocytic domain or a substance binding to the antigen on the surface of the immune effector cell into the immune effector cell. Since the killing effects of the substance on the immune effector cells after endocytosis are also completed in the cell membrane, the killing ability is remarkable. The present invention has been completed on this basis.

Fusion Protein and Immune Effector Cell of the Invention

To specifically kill immune effector cells, a fusion protein consisting of an antibody binding region, an optional linker moiety and an endocytic domain, i.e., a safety switch is expressed on the surface of an immune effector cell expressing a chimeric antigen receptor by the inventors. In the present invention, “the fusion protein of the present invention” has the same meaning as “safety switch”. In a specific embodiment, the immune effector cells include, but are not limited to, T cells or NK cells. Furthermore, as used herein, the term “active fragment” refers to a portion of a protein or polypeptide having an activity, i.e., the active fragment is not a full-length protein or polypeptide, but has the same or similar activity as the protein or polypeptide.

In a specific embodiment, the fusion protein of the present invention is as shown in Formula I

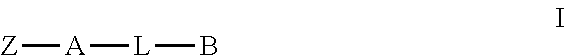

Wherein Z is an optional signal peptide;

A is an antibody binding region;

L is an optional linker moiety; and

B is an endocytic domain.

Based on the teachings of the present invention, a skilled person can think of and test various suitable linkers for being used in the fusion proteins of the present invention, which can be any suitable linker in the art, as long as the linker is capable of linking each part of the fusion protein of the invention and won't adversely affect the function of the resulting fusion protein. The optional linker means that a linker can be contained or not contained. Therefore, in a specific embodiment, the fusion protein of the present invention may comprise only the antibody binding region and the endocytic function region.

The fusion protein of the present invention binds to a specific antibody through an antibody binding region, and then the endocytic domain allows the fusion protein and antibody to be endocytosed into the immune cell. Thus, one of skill in the art can independently select an “antibody binding region” as described herein based on the teachings of the present invention. The antibody binding region in the fusion protein of the present invention is preferably a polypeptide which is not present in normal cells, or is in a concealed state in normal cells, or is low expressed in normal cells. For example, the antibody binding region epitope is an epitope in a concealed state in normal cells, including but not limited to normal cells expressing EGFR.

In a specific embodiment, the antibody may be, but is not limited to, an EGFR antibody, a GPC3 antibody, a mesothelin antibody, or the like, such as a CH12 antibody. The antibody binding region is selected from the following antigens or fragments thereof: EGFRvIII, EGFR, CD20, CD22, CD19, BCMA, proBDNF precursor protein, GPC3, CLD18.2, CLD6, mesothelin, PD-L1, PD-1, WT-1, IL13Ra2, Her-2, Her-1, Her-3; preferably, the antibody binding region comprises any one of the following amino acid sequences or comprises an amino acid sequence having at least 90%, 91%, 92%, 93%, 94%, 95%, 96%, 97%, 98% or 99% identity with the following amino acid sequence: SEQ ID NO: 28, 29, 33, 34, 35, 36, 37, 38, 39, 40, 41, 42, 43; more preferably, the antibody binding region comprises an active fragment of any one of the following amino acid sequences: SEQ ID NO: 28, 29, 33, 34, 35, 36, 37, 38, 39, 40, 41, 42, 43. In a specific embodiment, the antibody binding region specifically binds to an EGFR antibody.

The term “endocytic domain” as used herein refers to a functional moiety which, when the fusion protein binds to a specific binding substance of the antibody binding region, such as an antibody, will cause the fusion protein and the substance being endocytosed into the immune cell. The endocytic domain is derived from a folate receptor, LDL, CD30, CD33, CD3, EGFR, TFR1; preferably derived from a folate receptor and CD30; more preferably, the endocytic domain has an amino acid sequence of SEQ ID NO: 32 or 44, or an amino acid sequence having at least 90%, 91%, 92%, 93%, 94%, 95%, 96%, 97%, 98% or 99% identity with SEQ ID NO: 32 or 44, or is an active fragment of an amino acid sequence of SEQ ID NO: 32 or 44.

It is known to a skilled person that the signal peptide in the fusion protein of the present invention functions to help the fusion protein being pulled out of the cell membrane. Specific signal peptides can be determined by a skilled person. For example, the signal peptide can be a folate receptor signal peptide, a CD30 receptor signal peptide, a CD33 signal peptide, a CD8 signal peptide, preferably a folate receptor signal peptide. The signal peptide and endocytic domain in the fusion proteins of the present invention may be derived from the same or different proteins.

In a specific embodiment, the fusion protein of the present invention may have the amino acid sequence of SEQ ID NO: 10 or comprises an amino acid sequence having at least 90%, 91%, 92%, 93%, 94%, 95%, 96%, 97%, 98% or 99% identity with SEQ ID NO: 10 or an active fragment thereof.

Based on the teachings of the present invention, a skilled person will appreciate that the fusion protein of the present invention and a chimeric antigen receptor can be separately expressed or fusion-expressed on the surface of an immune effector cell. In a specific embodiment, the fusion protein of the present invention and the chimeric antigen receptor are separately expressed on the surface of an immune effector cell. As used herein, “separately expressed” means that the fusion protein and the chimeric antigen receptor are expressed on the surface of an immune effector cell, respectively, and the two are not in a fusion state; and “fusion-expressed” means that the fusion protein and the chimeric antigen are expressed in a form of fusion protein on the surface of an immune effector cells.

In a specific embodiment, the fusion protein of the present invention and the chimeric antigen receptor are fusion-expressed on the surface of an immune effector cell.

Based on the teachings of the present invention, a skilled person can select chimeric antigen receptors for different tumor antigens, for example, CD19-CAR, GPC3-CAR, CD30-CAR, Mesothelin-CAR, and the like. In a specific embodiment, a nucleotide sequence encoding the chimeric antigen receptor is shown in SEQ ID NO: 12. A skilled person can also use a technical means known in the art to promote fusion-expression of the fusion protein of the present invention and the chimeric antigen receptor on the surface of an immune effector cell, including but not limited to fusion-expression of the fusion protein and chimeric antigen receptor using self-cleaving sequences. In a specific embodiment, the self-cleaving sequence is preferably F2A or P2A. Among them, F2A is a core sequence derived from 2A of foot-and-mouth disease virus (or “self-cleaving polypeptide 2A”), and has a “self-cleaving” function of 2A, thereby achieving co-expression of upstream and downstream genes. 2A provides an effective and feasible strategy for constructing gene therapeutic polycistronic vectors due to its high cleaving efficiency, high balance of upstream and downstream gene expression and short self-sequence. In a preferred embodiment, the self-cleaving sequence is vkqtlnfdllklagdvesnpgp (SEQ ID NO: 30).

In a specific embodiment, the fusion protein of the present invention is shown in SEQ ID NO: 31.

The immune effector cell expressing the fusion protein of the present invention can achieve high-efficiency killing by using a specific antibody of the antibody binding region, and especially when the antibody binding region in the fusion protein is absent or in a concealed state in normal cells and a specific antibody of the antibody binding region is used to kill the immune effector cells, other normal cells won't be killed, thereby exhibiting excellent differential toxicity.

The immune effector cells of the present invention can be specifically killed by an immunoconjugate comprising: an antibody that specifically binds to an antibody binding region in the fusion protein of the present invention, and a cell-killing functional moiety. The cell-killing functional moiety comprises a cytotoxic molecule; preferably, the functional moiety is selected from the group consisting of MMAF, MMAE, Auristatin, calicheamicin, maytansine, maytansine, doxorubicin, paclitaxel, 5-fluorouracil, Methotrexate, DM1, DM4, MGBA and SN-38. The antibody and the cell-killing functional moiety may constitute a conjugate by covalent attachment, coupling, attachment, crosslinking, and the like.

A skilled person will appreciate that the antibody specifically binding to the antibody binding region in the fusion protein corresponds to the antibody binding region in the fusion protein of the present invention that is not present in normal cells. In a specific embodiment, the antibody specifically binding to the antibody binding region in the fusion protein is a CH12 antibody, but is not limited thereto. A skilled person can prepare the immunoconjugate with a suitable size based on the knowledge in the prior art, thereby facilitating endocytosis into the immune effector cells of the present invention for exerting killing effects.

A skilled person will appreciate that one particular form of the immunoconjugate is the antibody drug conjugate (ADC). After the antibody drug conjugate (ADC) enters a cell, the coupled toxic drug is released in an intracellular acidic environment and exerts toxic effects in the cell. Therefore, a receptor only having an endocytic domain on a cell binds to its corresponding antibody drug conjugate (ADC) and mediates endocytosis of the antibody drug conjugate (ADC). After the antibody drug conjugate (ADC) enters the cell, and the coupled toxic drug is released in an intracellular acidic environment, and exerts toxic effects in the cell.

Therefore, an immune effector cell expressing a chimeric antigen receptor is further provided in the present invention, the immune effector cell expresses an endocytic domain, and the endocytic domain is capable of transferring a substance binding to the endocytic domain into the immune effector cell. The substance is transferred into the immune effector cell to initiate killing of the immune effector cell. Thus, the endocytic domain described herein is capable of transferring a substance binding to the endocytic domain or a substance binding to the antibody binding region into the immune effector cell.

Preferably, the substance is an antibody drug conjugate (ADC). In a specific embodiment, the endocytic domain and the chimeric antigen receptor are separately expressed or fusion-expressed on the surface of the immune effector cell, preferably separately expressed.

Based on the fusion protein of the present invention, an encoding nucleic acid for the fusion protein of the present invention, an expression vector comprising the encoding nucleic acid and a host cell comprising the expression vector or having the encoding nucleic acid is integrated in its genome is further provided in the present invention.

The present invention also provides a kit comprising the immune effector cell or immunoconjugate of the present invention for treatment or killing of immune effector cells; that is, killing immune effector cells by administrating the immune conjugate of the present invention.

### Advantages of the Invention

1. The immune effector cell of the present invention can be recognized by a specific antibody, and can be killed by an antibody-conjugated drug derived from the antibody, and exhibits less influence on other normal cells, therefore having excellent differential toxicities;

2. The fusion protein expressed on the surface of the immune effector cell of the present invention is capable of causing the fusion protein and the antibody-conjugated drug to be endocytosed into the immune cell after binding to a specific antibody, thereby killing the immune effector cell by the coupled toxin molecule with powerful toxicity inside the cell membrane, therefore the killing ability is remarkable; and

3. The killing of immune effector cells by the technical solution of the present invention is mainly completed in cells, and is less affected by other factors (such as the complement system and NK cell activity in vivo on which CDC and ADCC depend), thereby killing immune effector cells expressing the fusion protein provided in the present application under various environments.

The invention is further illustrated below in conjunction with specific embodiments. It is to be understood that the examples are intended to demonstrate the invention while not intended to limit the scope of the invention. The experimental methods in the following examples, specific conditions of which are not specified are usually prepared according to conventional conditions such as conditions described in J. Sambrook et al., Molecular Cloning Experimental Guide, Third Edition, Science Press, 2002, or according to the conditions suggested by the manufacturer. For example, the flow analysis involved in the examples was performed using a Beckman flow analyzer, and the results were processed using FlowJo software. The materials used in the following examples are also commercially available.

### Example 1. Expression of Fusion Protein FR806

In this example, eGFP (enhanced green fluorescent protein) was selected as a fluorescent marker for analysis. F2A was selected as a self-cleaving sequence, and F2A is a core sequence derived from 2A of foot-and-mouth disease virus (or “self-cleaving polypeptide 2A”) and has a “self-cleaving” function of 2A; partial amino acid sequence (SEQ ID NO: 32) of human folate receptor of subtype 1 (FOLR1) and partial sequence of EGFR (SEQ ID NO: 28) were selected and expressed as a fusion protein FR806 (SEQ ID NO: 44); and the signal peptide of FOLR1 was selected. The following genetic engineering operations were performed using standard methods known to a skilled person. The nucleotide (SEQ ID NO: 1) of eGFP-F2A-FR806 was prepared as follows:

SEQ ID NO: 1

(eGFP is shown in bold, F2A is underlined, FR SP (folate receptor signal peptide) is shown in bold and underlined, 806 epitope is shown in italics, and the rest is the remaining part of folate receptor)

Atggtgagcaagggcgaggagctgttcaccggggtggtgcccatcctggt
cgagctggacggcgacgtaaacggccacaagttcagcgtgtccggcgagg
gcgagggcgatgccacctacggcaagctgaccctgaagttcatctgcacc
accggcaagctgcccgtgccctggcccaccctcgtgaccaccctgaccta
cggcgtcagtgcttcagccgctaccccgaccacatgaagcagcacgactt
cttcaagtccgccatgcccgaaggctacgtccaggagcgcaccatcttct
tcaaggacgacggcaactacaagacccgcgccgaggtgaagttcgagggc
gacaccctggtgaaccgcatcgagctgaagggcatcgacttcaaggagga
cggcaacatcctggggcacaagctggagtacaactacaacagccacaacg
tctatatcatggccgacaagcagaagaacggcatcaaggtgaacttcaag
atccgccacaacatcgaggacggcagcgtgcagctcgccgaccactacca
gcagaacacccccatcggcgacggccccgtgctgctgcccgacaaccact
acctgagcacccagtccgccctgagcaaagaccccaacgagaagcgcgat
cacatggtcctgctggagttcgtgaccgccgccgggatcactctcggcat
ggacgagctgtacaagtccggagtgaaacagactttgaattttgaccttc
tgaagttggcaggagacgttgagtccaaccctgggcccatggctcagcgg
atgacaacacagctgctgctccttctagtgtgggtggctgtagtagggga
ggctcagacagtccgagcctgtggggccgacagctatgagatggaggaag
acggcgtccgcaagtgtaagaagaggattgcatgggccaggactgagctt
ctcaatgtctgcatgaacgccaagcaccacaaggaaaagccaggccccga
ggacaagttgcatgagcagtgtcgaccctggaggaagaatgcctgctgtt
ctaccaacaccagccaggaagcccataaggatgtttcctacctatataga
ttcaactggaaccactgtggagagatggcacctgcctgcaaacggcattt
catccaggacacctgcctctacgagtgctcccccaacttggggccctgga
tccagcaggtggatcagagctggcgcaaagagcgggtactgaacgtgccc
ctgtgcaaagaggactgtgagcaatggtgggaagattgtcgcacctccta
cacctgcaagagcaactggcacaagggctggaactggacttcagggttta
acaagtgcgcagtgggagctgcctgccaacctttccatttctacttcccc
acacccactgttctgtgcaatgaaatctggactcactcctacaaggtcag
caactacagccgagggagtggccgctgcatccagatgtggttcgacccag
cccagggcaaccccaatgaggaggtggcgaggttctatgctgcagccatg
agtggggctgggccctgggcagcctggcctacctgcttagcctggcccta
atgctgctgtggctgctcagc

The amino acid sequence of eGFP-F2A-FR806 (SEQ ID NO: 2) is:

Mvskgeelftgvvpilveldgdynghkfsysgegegdatygkltlkfict
tgklpvpwptlyttltygvqcfsrypdhmkqhdffksampegyvqertif
fkddgnyktraevkfegdtlynrielkgidfkedgnilghkleynynshn
vyimadkqkngikvnfkirhniedgsvqladhyqqntpigdgpvllpdnh
ylstqsalskdpnelkdhmyllefvtaagitlgmdelyksgvkqtlnfdl
lklagdvesnpgpmaqrmttqlllllvwvavvgeaqtvracgadsyemee
dgvrkckkriawartellnvcmnakhhkekpgpedklheqcrpwrknacc
stntsqeahkdvsylyrfnwnhcgemapackrhfiqdtclyecspnlgpw
iqqvdqswrkervlnvplckedceqwwedcrtsytcksnwhkgwnwtsgf
nkcavgaacqpfhfyfptptvlcneiwthsykvsnysrgsgrciqmwfdp
aqgnpneevarfyaaamsgagpwaawpfllslalmllwlls

1. Preparation of Nucleotide Sequence of eGFP-F2A-FR806

1.1 Nucleotide sequences of FOLR1 signal peptide (SEQ ID NO: 3) and the rest of FOLR1 (SEQ ID NO: 4) were prepared according to the experimental procedure in J. Biol. Chem. 264: 14893-14901 (1989) and the sequence of Genebank Accession No. NM_016729.2.

SEQ ID NO: 3
Atggctcagcggatgacaacacagctgctgctccttctagtgtgggtggc
tgtagtaggggaggctcagaca
SEQ ID NO: 4
Aggattgcatgggccaggactgagcttctcaatgtctgcatgaacgccaa
gcaccacaaggaaaagccaggccccgaggacaagttgcatgagcagtgtc
gaccctggaggaagaatgcctgctgttctaccaacaccagccaggaagcc
cataaggatgtttcctacctatatagattcaactggaaccactgtggaga
gatggcacctgcctgcaaacggcatttcatccaggacacctgcctctacg
agtgctcccccaacttggggccctggatccagcaggtggatcagagctgg
cgcaaagagcgggtactgaacgtgcccctgtgcaaagaggactgtgagca
atggtgggaagattgtcgcacctcctacacctgcaagagcaactggcaca
agggctggaactggacttcagggtttaacaagtgcgcagtgggagctgcc
tgccaacctttccatttctacttccccacacccactgttctgtgcaatga
aatctggactcactcctacaaggtcagcaactacagccgagggagtggcc
gctgcatccagatgtggttcgacccagcccagggcaaccccaatgaggag
gtggcgaggttctatgctgcagccatgagtggggctgggccctgggcagc
ctggcctttcctgcttagcctggccctaatgctgctgtggctgctcagc

The nucleotide sequence of position 284-304 epitope of EGFR was prepared according to the experimental procedure in Journal of Biological Chemistry, 2004, 279(29), 30375-30384 and the sequence of Genebank Accession No. X00588.1 (SEQ ID NO: 5).

SEQ ID NO: 5
Gtccgagcctgtggggccgacagctatgagatggaggaagacggcgtccg
caagtgtaagaag

The nucleotide sequence of SEQ ID NO: 3, the nucleotide sequence of SEQ ID NO: 4 and the nucleotide sequence of SEQ ID NO: 5 were combined in order, and then Suzhou Jinweizhi Biotechnology Co., Ltd. was entrusted to complete the synthesis of whole gene combination, so as to obtain a gene fragment of the nucleotide sequence of FR806 (SEQ ID NO: 6).

SEQ ID NO: 6
Atggctcagcggatgacaacacagctgctgctccttctagtgtgggtggc
tgtagtaggggaggctcagacagtccgagcctgtggggccgacagctatg
agatggaggaagacggcgtccgcaagtgtaagaagaggattgcatgggcc
aggactgagcttctcaatgtctgcatgaacgccaagcaccacaaggaaaa
gccaggccccgaggacaagttgcatgagcagtgtcgaccctggaggaaga
atgcctgctgttctaccaacaccagccaggaagcccataaggatgtttcc
tacctatatagattcaactggaaccactgtggagagatggcacctgcctg
caaacggcatttcatccaggacacctgcctctacgagtgctcccccaact
tggggccctggatccagcaggtggatcagagctggcgcaaagagcgggta
ctgaacgtgcccctgtgcaaagaggactgtgagcaatggtgggaagattg
tcgcacctcctacacctgcaagagcaactggcacaagggctggaactgga
cttcagggittaacaagtgcgcagtgggagctgcctgccaacctttccat
ttctacttccccacacccactgttctgtgcaatgaaatctggactcactc
ctacaaggtcagcaactacagccgagggagtggccgctgcatccagatgt
ggttcgacccagcccagggcaaccccaatgaggaggtggcgaggttctat
gctgcagccatgagtggggctgggccctgggcagcctggcctttcctgct
tagcctggccctaatgctgctgtggctgctcagc

1.2. In order to obtain an eGFP nucleic acid fragment containing F2A (66 bp) at 3′ end and a small nucleic acid (20 bp) assembled downstream, pWPT-eGFP-F2A-GPC3-BBZ used in CN201310164725.X was used as a template (see SEQ ID NO: 28 in CN201310164725.X).

PCR amplification was carried out with upstream primer 5′-gcaggggaaagaatagtagaca-3′ (SEQ ID NO: 7) and downstream primer 5′-gttgtcatccgctgagccatgggcccagggttggactc-3′ (SEQ ID NO: 8) to obtain an eGFP nucleic acid fragment containing F2A (66 bp) at 3′ end and a small nucleic acid (20 bp) assembled downstream.

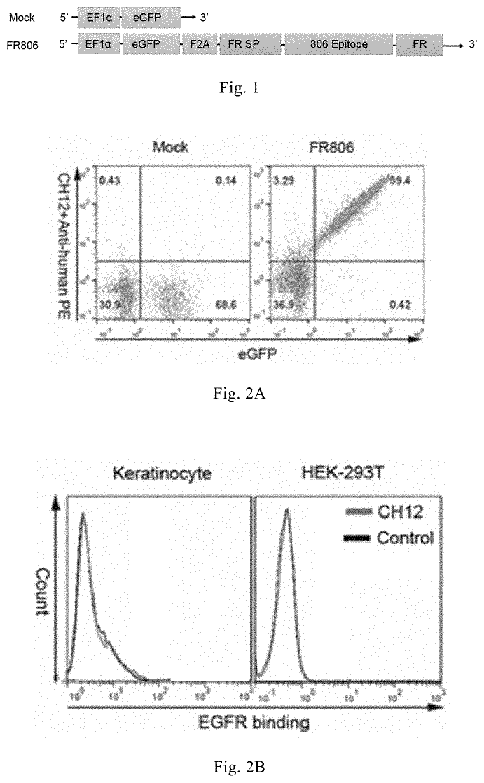

1.3 Equimolar amount of the eGFP nucleic acid fragment containing F2A (66 bp) at 3′ end and a small nucleic acid (20 bp) assembled downstream obtained in step 1.2 and the FR806 nucleotide sequence fragment obtained in step 1.1 were linked and subjected to PCR according to the manner as shown in FIG. 1. In FIG. 1, FR SP represents the signal peptide of folate receptor (SEQ ID NO: 3), 806 epitope represents EGFR284-304 epitope (SEQ ID NO: 5), and FR represents other parts of folate receptor except signal peptide (SEQ ID NO: 4). The DNA polymerase was supplemented, and the upstream primer 5′-gcaggggaaagaatagtagaca-3′ (SEQ ID NO: 7) and the downstream primer 5′-ctcgaggtcgacctagctgagcagccacagc-3′ (SEQ ID NO: 9) were added and subjected to PCR to obtain gene fragments of the nucleotide sequence of eGFP-F2A-FR806 containing Mul I Sal I cleavage sites at both ends, the theoretical size of which is 2047 bp, and the amplified product was confirmed by agarose gel electrophoresis to be in agreement with the theoretical size.

2. Construction of eGFP-F2A-FR806 Lentiviral Vector

The vector system used in the lentiviral plasmid vector used in this example belongs to the third generation of auto-inactivated lentiviral vector system, and the system comprises: packaging plasmid psPAX2 encoding protein Gag/Pol, Rev protein, envelope plasmid PMD2.G encoding VSV-G protein and a recombinant expression vector encoding the target gene eGFP-F2A-FR806 based on empty vector pWPT-eGFP.

In the empty vector pWPT-eGFP, the promoter of elongation factor-1α (elongation factor-1α, EF-1α) regulates the expression of enhanced green fluorescent protein (eGFP), while in the recombinant expression vector encoding the target gene eGFP-F2A-FR806, eGFP was co-expressed with the target gene FR806 by a ribosomal skipping sequence of food and mouth disease virus (FMDV, F2A).

The gene fragments of the nucleotide sequence of eGFP-F2A-FR806 containing Mul I Sal I cleavage sites at both ends obtained in example 1.1 were digested by MluI and SalI restriction enzymes, and ligated into pWPT vector which was also double-digested, so as to construct a plasmid pWPT-eGFP-F2A-FR806 co-expressing eGFP and FR806 linked by F2A.

3. Lentivirus Packaging and Concentration

293T cells (ATCC) were inoculated in a 15 cm culture dish at a density of 1.25×10⁷ in L110 DMEM medium (Gbico) containing 10% fetal bovine serum (Gbico).

27.5 μg of pWPT-eGFP-F2A-FR806 plasmid, 27.5 μg of pWPT-eGFP (Mock) control plasmid, 20.7 μg of packaging plasmid PAX2 and 8.3 μg of envelope plasmid pMD2.G were dissolved in 2200 ul of serum-free DMEM medium, 165 μg of PEI (polyscience) was dissolved in 2200 ul of serum-free DMEM medium, and both of them were mixed and added to 293T. After 72 hours, the supernatant containing the virus was collected for filtration, and the virus was concentrated after purification.

4. Transduction of T Lymphocytes by Lentivirus

Human peripheral blood mononuclear cells were added to the lymphocyte culture medium at a density of about 1×10⁶/mL, and magnetic beads coated with anti-CD3 and anti-CD28 antibodies were added at a magnetic bead:cell ratio of 1:1 (Invitrogen) and recombinant human IL-2 (Shanghai Huaxin Biotech Co., Ltd.) was added at a final concentration of 300 U/mL for activation for 48 h.

The activated T cells were added to a plate (24-well plate) coated with Retronectin (purchased from takara) at a concentration of 1×10⁶ cells/ml, and the virus concentrate (MOI≈10) obtained in step 3 was added thereto, centrifuged and cultured in an incubator to obtain T cells (CAR-FR806-T cells) expressing fusion proteins FR806 and eGFP and Mock T cells, wherein the sequence of FR806 fusion protein further contains a signal peptide, as shown in SEQ ID NO: 10.

SEQ ID NO: 10
Maqrmttqlllllvwvavvgeaqtvracgadsyemeedgvrkckkriawa
rtellnvcmnakhhkekpgpedklheqcrpwrknaccstntsqeahkdvs
ylyrfnwnhcgemapackrhfiqdtclyecspnlgpwiqqvdqswrkerv
lnvplckedceqwwedcrtsytcksnwhkgwnwtsgfnkcavgaacqpfh
fyfptptvlcneiwthsykvsnysrgsgrciqmwfdpaqgnpneevarfy
aaamsgagpwaawpfllslalmllwlls

5. Detection of Expression of Fusion Receptor FR806 and eGFP in T Cells Through Flow Cytometry

CAR-FR806-T cells and Mock T cells obtained in step 4 were taken. The primary antibody, CH12 antibody (10 μg/ml) as disclosed in CN 200810038848.8 was incubated for 45 min, followed by washing with 1% FBS in PBS twice. The secondary antibody was PE-labeled goat anti-human IgG (Santa), incubated for 45 min at 1:50 dilution, washed twice with 1% FBS in PBS, resuspended, and subjected to flow analysis. The results are shown in FIG. 2A, indicating that T cells expressing FR806 fusion protein can effectively bind to CH12 antibody, and can be co-expressed with eGFP in T cells. The light chain of CH12 antibody is set forth in SEQ ID NO: 46 and the heavy chain is set forth in SEQ ID NO: 45.

Keratinocyte cells and HEK-293T cells expressing EGFR were selected, and the binding of CH12 antibody to both was analyzed by FACS. The results showed that CH12 antibody did not bind to both of EGFR-expressing Keratinocyte cells and HEK-293T cells (FIG. 2B).

### Example 2. Synthesis and Titration of CH12-Biotin

CH12 antibody was labeled with biotin. CH12 antibody was diluted to 2.5 mg/ml in PBS pH 7.4, and the labeled volume was 1.6 ml; 1 mg of Sulfo-NHS-LC-Biotin (Thermo) was taken and dissolved in 180 ul of ultrapure water; 79 ul of Biotin was added to 1.6 ml of CH12 antibody overnight. The mixture was desalted using a PD-10 desalting column (GE Corporation, USA), and replaced with 5% glycerol buffer in PBS to obtain CH12-Biotin, and the concentration was determined as 0.77 mg/ml at OD280/1.45.

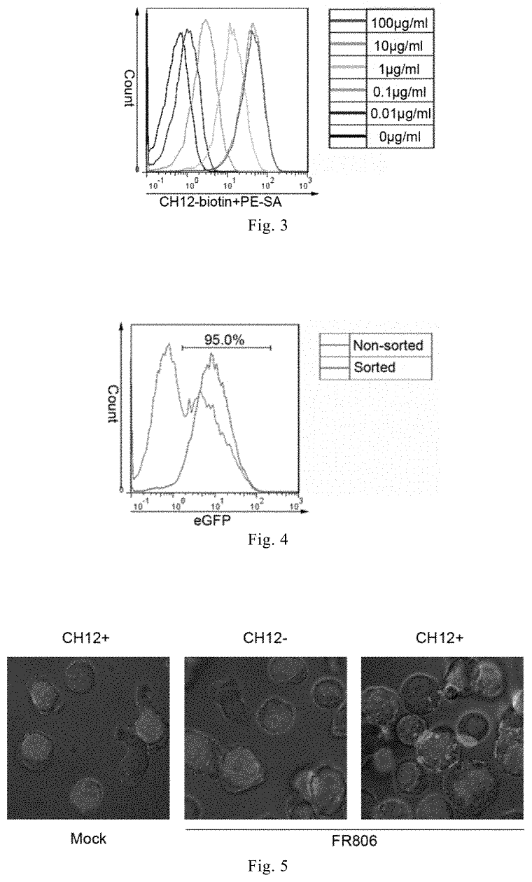

CH12-biotin was diluted to different concentrations (100 μg/ml, 10 μg/ml, 1 μg/ml, 0.1 μg/ml, 0.01 μg/ml, 0 μg/ml) in PBS containing 1% FBS, incubated with T cells expressing eGFP-F2A-FR806 for 45 min, and washed by PBS. The secondary antibody, PE-SA (ebioscience) was diluted at 1:300 in the medium, and resuspended cells were added and incubated for 45 min. Cells were washed twice with PBS and subjected to flow analysis. The results of flow analysis are showed in FIG. 3, demonstrating that the higher the concentration of CH12-biotin, the stronger the affinity, and the binding level at 10 μg/ml was similar to that at 100 μg/ml.

### Example 3. Sorting FR806-Positive T Cells with CH12-Biotin

1×10⁷ T cells expressing eGFP-F2A-FR806 were taken, washed with PBS, incubated with CH12-biotin (10 μg/ml, diluted with PBS containing 1% FBS) for 45 min at 4° C. and washed with PBS. Anti-Biotin sorting beads (purchased from Meitian Company) were added. T cells expressing FR806 were sorted according to the procedure provided with the sorting magnetic bead. Suitable amounts of the cells before and after sorting were taken and subjected to flow analysis. The results are shown in FIG. 4, demonstrating that, after binding to CH12-biotin, the T cells expressing FR806 can be effectively sorted by anti-Biotin sorting magnetic beads, and the positive rate of sorting is up to 95%.

### Example 4. Endocytosis Experiment of T Cells Expressing FR806

T cells infected with the lentiviral vectors pWPT-eGFP-F2A-FR806 and pWPT-eGFP (Mock) obtained in Example 1 were taken and washed with PBS; CH12-biotin synthesized in Example 2 (10 μg/ml, diluted in the medium) was taken, the secondary antibody was PE-SA (ebioscience) diluted at 1:300 in the medium, and resuspended cells were added and incubated for 45 min. Cells were washed twice with PBS, incubated for 4 h, afterwards, fixed in paraformaldehyde, stained with DAPI staining solution (Roche) and observed under a confocal microscope. The results are shown in FIG. 5. In the T cells expressing FR806, CH12-biotin (represented by red fluorescence) appeared inside the cell membrane, demonstrating that it can be effectively endocytosed by T cells.

### Example 5: Synthesis and Determination of Cell Killing Activities of Antibody-Conjugated Drug CH12-MMAF

1 ml (0.033 mM) of CH12 antibody was taken, into which 10 ul of DTPA (Thermo) and 1 ul of 100 mM TCEP (Thermo) were added, and MMAF in DMSO (concentration 3.4 mM) was added at a ratio of antibody:MMAF=10:1 at 4° C. for 3 h. The excess of MMAF was removed to obtain the antibody-conjugated drug CH12-MMAF.

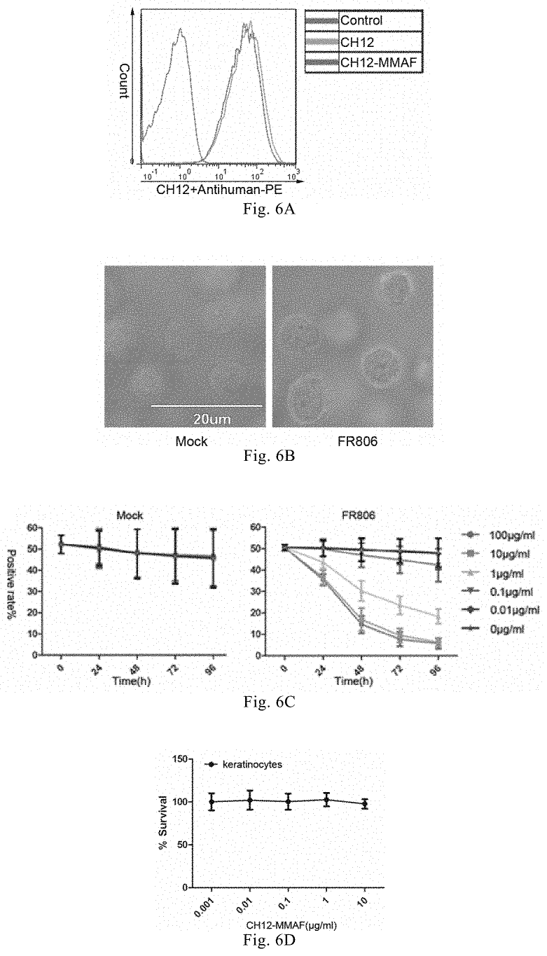

The ability of CH12 antibody and CH12-MMAF to bind to FR806-expressing T cells was detected by flow cytometry, and the results are shown in FIG. 6A.

According to the procedure of Example 4, T cells infected with pWPT-eGFP-F2A-FR806 and pWPT-eGFP (Mock) were taken and washed with PBS. CH12-MMAF (10 μg/ml, diluted in culture medium) was taken and incubated at 4° C. for 45 min and washed with PBS. The second antibody was goat anti-human PE (Shanghai Lianke Biotechnology Co., Ltd.) diluted at 1:50, and resuspended cells were added and incubated for 45 min. Cells were washed twice with PBS, incubated for 4 h, afterwards, fixed in paraformaldehyde, diluted in DAPI staining solution (Roche) at 1:500, stained with the second antibody for 2 min and observed under a confocal microscope. The results are shown in FIG. 6B, demonstrating that CH12-MMAF was able to be endocytosed by T cells expressing FR806.

The positive rates of T cells infected with Mock and eGFP-FR806 were detected by flow cytometry, and then the positive rates of T cells of Mock (control group) and eGFP-FR806 (experimental group) were adjusted to 50% by adding appropriate proportion of uninfected T cells. T cells were plated in 6-well plates at 2×10⁶ cells per well in 2 ml medium (AIM-V PBS+2% human AB serum, IL-2 500 U/ml). CH12-MMAF drugs were diluted to 0.01, 0.1, 1, 10 and 100 μg/ml with PBS respectively, and then added to the experimental group and the control group. The eGFP positive rate was detected every 24 hours for 96 hours. The results are shown in FIG. 6C, demonstrating that, after addition of CH12-MMAF, there T cells expressing FR806 were significantly killed, and the killing of FR806-expressing T cells was enhanced with the increase of CH12-MMAF concentration, wherein, at a dosage of 10 μg/ml and at 96 h, the killing effect on T cells expressing FR806 was up to 88%. For T cells (Mock) not expressing FR806, CH12-MMAF exhibits no killing effects, indicating that CH12-MMAF is safe.

The killing effects of CH12-MMAF on human Keratinocy cells were examined. As shown in FIG. 6D, CH12-MMAF did not kill human Keratinocy cells, indicating that CH12-MMAF was safe.

### Example 6. CCK8 Assay for Killing FR806-Expressing T Cells by CH12-MMAF Drug and Free MMAF

Experimental group: T cells expressing eGFP-FR806 after sorted in Example 3 were plated in a 96-well plate at 3×10⁴ cells per well in 100 ul of medium, 5 replicate wells per drug concentration, and then a blank group of medium was set. Control group: T cells that were not infected with the virus were taken and plated in a 96-well plate according to the operation of the experimental group. CH12-MMAF at concentrations of 100 μg/ml, 10 μg/ml, 1 μg/ml, 0.1 μg/ml, 0.01 μg/ml, and 0 μg/ml were taken and added to the T cells of the experimental group and the control group, respectively, to prepare six gradients (i.e., six concentrations of 100 μg/ml, 10 μg/ml, 1 μg/ml, 0.1 μg/ml, 0.01 μg/ml, 0 μg/ml as said above). After 72 h, 10 ul of CCK8 reagent (Dojindo) was added to each well and incubated at 37° C. for 3 h, and the absorbance was measured at 450 nm by a microplate reader to calculate the cell viability.

According to the above procedure, the sorted T cells infected with eGFP-FR806 were taken and plated in a 96-well plate at 3×10⁴ cells per well in 100 ul of culture medium, 5 replicate wells per drug concentration, and then a blank group of medium was set. The control group was uninfected T cells, which were plated in a 96-well plate by the same method. Six concentrations of free MMAFs of 1000 nM, 500 nM, 100 nM, 50 nM, 10 nM and 0 nM were added to T cells at specific concentrations to prepare six gradients (i.e., the aforementioned six concentrations). After 72 h, 10 ul of CCK8 reagent (Dojindo) was added to each well for 3 h at 37° C., and the absorbance was measured at 450 nm by a microplate reader to calculate the cell viability.

The calculation formula is: cell viability (%)=[A (dosing)−A (blank)]/[A (0 dosing)−A (blank)]

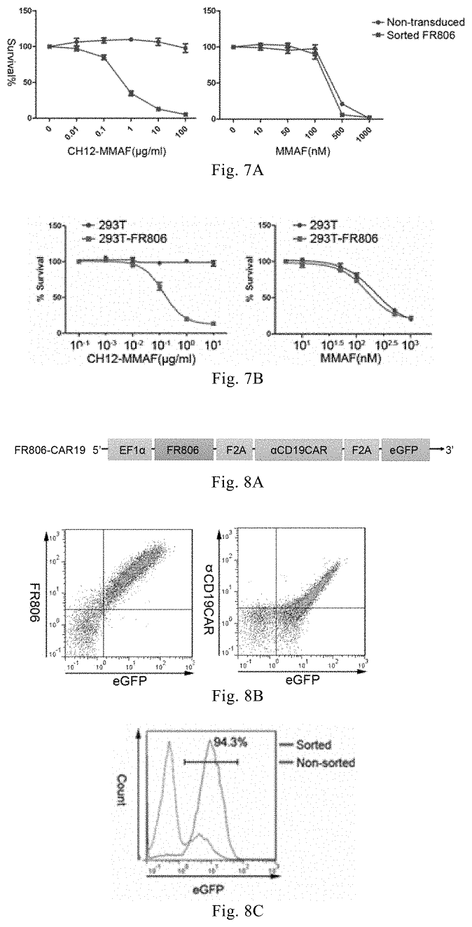

The results are shown in FIG. 7A, showing that CH12-MMAF specifically kills FR806-positive T cells. The killing levels of Free MMAF to T cells expressing and not expressing FR806 are comparable.

Moreover, the applicant selected EGFR+ HEK293T cells expressing FR806, and subjected them to cell killing experiments. The results are shown in FIG. 7B, demonstrating that CH12-MMAF significantly killed FR806-positive HEK293T, while not obviously killed FR806-negative HEK293T, and MMAF killed both of FR806 positive and negative HEK293T. It is indicated that even if the cells are EGFR positive, CH12-MMAF won't kill the cells as long as they do not express FR806.

### Example 7. Preparation of FR806-CAR19 T Cells

In this example, eGFP was selected as a fluorescent marker, and eGFP was enhanced green fluorescent protein. The following genetic engineering operations were performed using standard methods known to a skilled person.

In this example, the nucleotide fragment of single-chain antibody of αCD19 disclosed in US20060193852A1 (SEQ ID NO: 11) was selected as the anti-CD19 antibody sequence of CAR, and CD8-CD137-CD3 was selected as the transmembrane domain and intracellular domain of CAR.

SEQ ID NO: 11
gatatccagctgacccagtctccagcttctttggctgtgtctctagggca
gagggccaccatctcctgcaaggccagccaaagtgttgattatgatggtg
atagttatttgaactggtaccaacagattccaggacagccacccaaactc
ctcatctatgatgcatccaatctagtttctgggatcccacccaggtttag
tggcagtgggtctgggacagacttcaccctcaacatccatcctgtggaga
aggtggatgctgcaacctatcactgtcagcaaagtactgaggatccgtgg
acgttcggtggagggaccaagctcgagatcaaaggtggtggtggttctgg
cggcggcggctccggtggtggtggttctcaggtgcagctgcagcagtctg
gggctgagctggtgaggcctgggtcctcagtgaagatttcctgcaaggct
tctggctatgcattcagtagctactggatgaactgggtgaagcagaggcc
tggacagggtcttgagtggattggacagataggcctggagatggtgatac
taactacaatggaaagttcaagggtaaagccactctgactgcagacgaat
cctccagcacagcctacatgcaactcagcagcctagcatctgaggactct
gcggtctatttctgtgcaagacgggagactacgacggtaggccgttatta
ctatgctatggactactggggccaagggaccacggtcaccgtctcctcc

1. Preparation of Nucleotide Sequence of FR806-F2A-CAR(CD19)-F2A-eGFP

1.1 αCD19CAR nucleotide sequence with a partial F2A fragment at 3′ and 5′ ends, respectively

Suzhou Jinweizhi Biotechnology Co., Ltd. was entrusted to carry out the whole genome synthesis to obtain the gene fragment of the nucleotide sequence of αCD19CAR (SEQ ID NO: 12), the nucleotide fragment containing CD8α signal peptide sequence, the single-chain antibody of αCD19 and CD8-CD137-CD3ζ nucleic acid fragment containing a sequence of a hinge region, a transmembrane region and an intracellular segment.

SEQ ID NO: 12 (CD8α signal peptide sequence is shown in bold, αCD19CAR nucleotide sequence is underlined, and CD8-CD137-CD3ζ nucleotide sequence is shown in italics and bold)

atggccttaccagtgaccgccttgctcctgccgctggccttgctgctccacgccgccaggccggatatccagctgacccagtctc
cagcttctttggctgtgtctctagggcagagggccaccatctcctgcaaggccagccaaagtgttgattatgatggtgatagttatttgaactggta
ccaacagattccaggacagccacccaaactcctcatctatgatgcatccaatctagtttctgggatcccacccaggtttagtggcagtgggtctg
ggacagacttcaccctcaacatccatcctgtggagaaggtggatgctgcaacctatcactgtcagcaaagtactgaggatccgtggacgttcg
gtggagggaccaagctcgagatcaaaggtggtggtggttctggcggcggcggctccggtggtggtggttctcaggtgcagctgcagcagtct
ggggctgagctggtgaggcctgggtcctcagtgaagatttcctgcaaggcttctggctatgcattcagtagctactggatgaactgggtgaagc
agaggcctggacagggtcttgagtggattggacagatttggcctggagatggtgatactaactacaatggaaagttcaagggtaaagccactct
gactgcagacgaatcctccagcacagcctacatgcaactcagcagcctagcatctgaggactctgcggtctatttctgtgcaagacgggagac
tacgacggtaggccgttattactatgctatggactactggggccaagggaccacggtcaccgtctcctcc

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

1.2 the gene fragment of the nucleotide sequence of the synthesized αCD19CAR (SEQ ID NO: 12) was used as a template, and the primer pair for amplification was the upstream primer 5′-ccttctgaagttggcaggagacgttgagtccaaccctgggcccatggccttaccagtg-3′ (SEQ ID NO: 13) and downstream primer 5′-tcctgccaacttcagaaggtcaaaattcaaagtctgtttcacgcgagggggcagggc-3′ (SEQ ID NO: 14), so as to obtain αCD19CAR nucleotide sequence with a partial F2A fragment at 3′ and 5′ ends, respectively. The PCR amplified bands were determined by agarose gel electrophoresis to match the expected fragment size.

2. Preparation of Nucleic Acid Sequence of FR806-F2A-CAR19-F2A-eGFP

To prepare the linking sequence FR806-F2A-CAR19-F2A-eGFP (SEQ ID NO: 15) of FR806, αCD19CAR and eGFP, the following procedure was used:

SEQ ID NO: 15 (FR806 is underlined, αCD19CAR is shown in bold and underlined, F2A is shown in bold, and eGFP is normally displayed)

atggctcagcggatgacaacacagctgctgctccttctagtgtgggtggc
tgtagtaggggaggctcagacagtccgagcctgtggggccgacagctatg
agatggaggaagacggcgtccgcaagtgtaagaagaggattgcatgggcc
aggactgagcttctcaatgtctgcatgaacgccaagcaccacaaggaaaa
gccaggccccgaggacaagttgcatgagcagtgtcgaccctggaggaaga
atgcctgctgttctaccaacaccagccaggaagcccataaggatgtttcc
tacctatatagattcaactggaaccactgtggagagatggcacctgcctg
caaacggcatttcatccaggacacctgcctctacgagtgctcccccaact
tggggccctggatccagcaggtggatcagagctggcgcaaagagcgggta
ctgaacgtgcccctgtgcaaagaggactgtgagcaatggtgggaagattg
tcgcacctcctacacctgcaagagcaactggcacaagggctggaactgga
cttcagggtttaacaagtgcgcagtgggagctgcctgccaacctttccat
ttctacttccccacacccactgttctgtgcaatgaaatctggactcactc
ctacaaggtcagcaactacagccgagggagtggccgctgcatccagatgt
ggttcgacccagcccagggcaaccccaatgaggaggtggcgaggttctat
gctgcagccatgagtggggctgggccctgggcagcctggcctttcctgct
tagcctggccctaatgctgctgtggctgctcagcgtgaaacagactttga
attttgaccttctgaagttggcaggagacgttgagtccaaccctgggccc
atggccttaccagtgaccgccttgctcctgccgctggccttgctgctcca
cgccgccaggccggatatccagctgacccagtctccagcttctttggctg
tgtctctagggcagagggccaccatctcctgcaaggccagccaaagtgtt
gattatgatggtgatagttatttgaactggtaccaacagattccaggaca
gccacccaaactcctcatctatgatgcatccaatctagtttctgggatcc
cacccaggtttagtggcagtgggtctgggacagacttcaccctcaacatc
catcctgtggagaaggtggatgctgcaacctatcactgtcagcaaagtac
tgaggatccgtggacgttcggtggagggaccaagctcgagatcaaaggtg
gtggtggttctggcggcggcggctccggtggtggtggttctcaggtgcag
ctgcagcagtctggggctgagctggtgaggcctgggtcctcagtgaagat
ttcctgcaaggcttctggctatgcattcagtagctactggatgaactggg
tgaagcagaggcctggacagggtcttgagtggattggacagatttggcct
ggagatggtgatactaactacaatggaaagttcaagggtaaagccactct
gactgcagacgaatcctccagcacagcctacatgcaactcagcagcctag
catctgaggactctgcggtctatttctgtgcaagacgggagactacgacg
gtaggccgttattactatgctatggactactggggccaagggaccacggt
caccgtctcctccaccacgacgccagcgccgcgaccaccaacaccggcgc
ccaccatcgcgtcgcagcccctcccctgcgcccagaggcgtgccggccag
cggcggggggcgcagtgcacacgagggggctggacttcgcctgtgatatc
tacatctgggcgcccttggccgggacttgtggggtccttctcctgtcact
ggttatcaccctttactgcaaacggggcagaaagaaactcctgtatatat
tcaaacaaccatttatgagaccagtacaaactactcaagaggaagatggc
tgtagctgccgatttccagaagaagaagaaggaggatgtgaactgagagt
gaagttcagcaggagcgcagacgcccccgcgtaccagcagggccagaacc
agctctataacgagctcaatctaggacgaagagaggagtacgatgttttg
gacaagagacgtggccgggaccctgagatggggggaaagccgcagagaag
gaagaaccctcaggaaggcctgtacaatgaactgcagaaagataagatgg
cggaggcctacagtgagattgggatgaaaggcgagcgccggaggggcaag
gggcacgatggcctttaccagggtctcagtacagccaccaaggacaccta
cgacgcccttcacatgcaggccctgccccctcgcgtgaaacagactttga
attttgaccttctgaagttggcaggagacgttgagtccaaccctgggccc
atggtgagcaagggcgaggagctgttcaccggggtggtgcccatcctggt
cgagctggacggcgacgtaaacggccacaagttcagcgtgtccggcgagg
gcgagggcgatgccacctacggcaagctgaccctgaagttcatctgcacc
accggcaagctgcccgtgccctggcccaccctcgtgaccaccctgaccta
cggcgtgcagtgcttcagccgctaccccgaccacatgaagcagcacgact
tcttcaagtccgccatgcccgaaggctacgtccaggagcgcaccatcttc
ttcaaggacgacggcaactacaagacccgcgccgaggtgaagttcgaggg
cgacaccctggtgaaccgcatcgagctgaagggcatcgacttcaaggagg
acggcaacatcctggggcacaagctggagtacaactacaacagccacaac
gtctatatcatggccgacaagcagaagaacggcatcaaggtgaacttcaa
gatccgccacaacatcgaggacggcagcgtgcagctcgccgaccactacc
agcagaacacccccatcggcgacggccccgtgctgctgcccgacaaccac
tacctgagcacccagtccgccctgagcaaagaccccaacgagaagcgcga
tcacatggtcctgctggagttcgtgaccgccgccgggatcactctcggca
tggacgagctgtacaag

2.1, the eGFP-F2A-FR806 lentiviral vector constructed in Example 1 was used as a template for PCR amplification, and the primer pair for amplification was the upstream primer 5′-cttacgcgtcctagcgctaccggtcgccaccatggctcagcggatg-3′ (SEQ ID NO: 16) and downstream primer 5′-gtctcctgccaacttcagaaggtcaaaattcaaagtctgtttcacgctgagcagccac-3′ (SEQ ID NO: 17). The size of the amplified band was 910 bp. The PCR amplification conditions were pre-denaturation: 94° C., 4 min; denaturation: 94° C., 40 s; annealing: 58° C., 40 s; extension: 68° C., 1 min; 25 cycles followed by a total extension of 68° C., 10 min. The PCR-amplified bands were determined by agarose gel electrophoresis to determine the size of the amplified bands of interest.

2.2 Amplification of eGFP-F2A-FR806 sequence with a partial F2A fragment at 5′ end

the eGFP-F2A-FR806 lentiviral vector constructed in Example 2 was used as a template, and the primer pair for amplification was the upstream primer 5′-accttctgaagttggcaggagacgttgagtccaaccctgggcccatggtgagcaagggc-3′ (SEQ ID NO: 18) and the downstream primer 5′-ctcgaggtcgacctacttgtacagctcg-3′ (SEQ ID NO: 19), so as to obtain eGFP-F2A-FR806 nucleic acid fragment with a partial F2A fragment at 5′ end. The PCR-amplified bands were determined by agarose gel electrophoresis to match the expected fragment size.

2.3. Equimolar amount of the nucleotide sequences of αCD19CAR having a partial F2A fragment at 3′ and 5′ ends, respectively, and the FR806 sequence having a partial F2A fragment at 3′ end were linked and subjected to PCR according to the manner as shown in FIG. 8A. The DNA polymerase was supplemented, and the upstream primer 5′-cttacgcgtcctagcgctaccggtcgccaccatggctcagcggatg-3′(SEQ ID NO: 16) and the downstream primer 5′-tcctgccaacttcagaaggtcaaaattcaaagtctgtttcacgcgagggggcagggc-3′ (SEQ ID NO: 14) were added and subjected to PCR for 25 cycles to obtain linked fragments of FR806 and αCD19CAR nucleotide sequences, the theoretical size of which is 2458 bp, and the amplified product was confirmed by agarose gel electrophoresis to be in agreement with the theoretical size.

2.4. Equimolar amount of linked fragments of the nucleotide sequences of FR806 and αCD19CAR and eGFP sequence having a partial F2A fragment at 5′ end were linked and subjected to PCR according to the manner as shown in FIG. 8A. The DNA polymerase was supplemented, and the upstream primer 5′-cttacgccccctagcgcccccggtcgccaccatggctcagcggatg-3′ (SEQ ID NO: 16) and the downstream primer 5′-ctcgaggtcgacctacttgtacagctcg-3′ (SEQ ID NO: 19) were added and subjected to PCR for 25 cycles to obtain a linked fragment FR806-F2A-CAR19-F2A-eGFP of FR806 and αCD19CAR as well as eGFP, the theoretical size of which is 32148 bp, and the amplified product was confirmed by agarose gel electrophoresis to be in agreement with the theoretical size.

3. Construction of FR806-F2A-CAR19-F2A-eGFP Lentiviral Vector

According to the construction of the lentiviral vector in Example 1, the obtained nucleotide sequence of FR806-F2A-CAR19-F2A-eGFP was digested with MluI and SalI restriction enzymes and ligated into pWPT vector which was also double-digested, so as to construct a F2A-linked lentiviral expression vector co-expressing FR806, αCD19CAR and eGFP.

4. Plasmid Transfection 293T Packaging Lentivirus

According to the operation of step 3 in Example 1, the lentiviral expression vector obtained in step 2 of the present example, pWPT-eGFP control plasmid, the packaging plasmid PAX2 and envelope plasmid pMD2.G were dissolved in 2200 ul of serum-free DMEM medium for lentiviral packaging.

5. Lentivirus-Transduction of T Cells

According to the operation of step 4 in Example 1, the packaged lentivirus obtained in step 3 of the present example was transfected into T cells to obtain CAR-T cells with surface-expressed CAR19 and FR806, namely FR806-CAR19 T cells, and FR806-CAR19 T cells were subjected to flow analysis. The results are shown in FIG. 8B, demonstrating that the three proteins FR806, eGFP and αCD19CAR can be efficiently expressed in T cells.

According to the operation in Example 3, FR806-CAR19 T cells were sorted using CH12-biotin and anti-biotin magnetic beads. The results are shown in FIG. 8C, demonstrating that FR806-CAR19 T cells, after binding to CH12-biotin, can be effectively sorted with anti-Biotin sorting magnetic beads, and the positive rate of sorting was 94.3%.

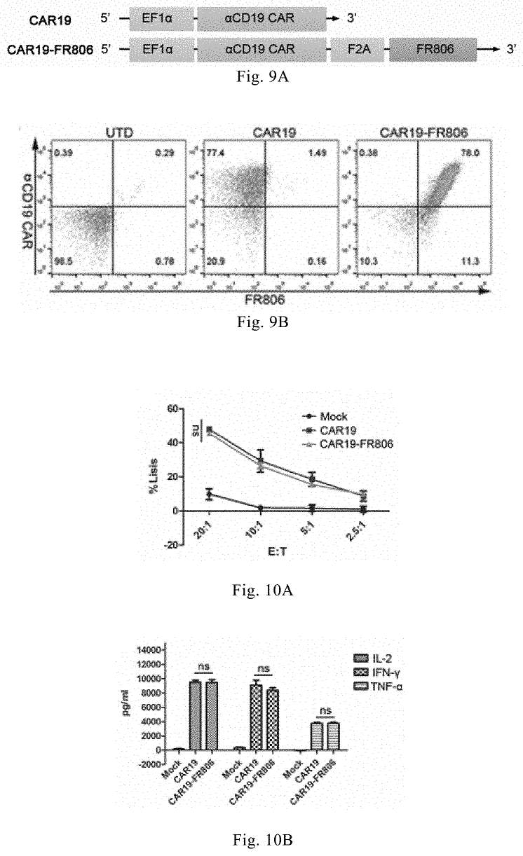

According to the above operations, linking and PCR were carried out in accordance with the mode shown in FIG. 9A to obtain T cells (FR806-CAR19 T cells) expressing FR806 and CAR19. The T cells were subject to flow cytometry, and the results are shown in FIG. 9B.

### Example 8. Killing Effects of FR806-CAR19 T Cell on Tumor Cells and Cytokine Release

According to the operation in Example 7, T cells expressing CAR19 and not expressing FR806, namely CAR19 T cells, were obtained. The resulting FR806-CAR19 T cells linked and obtained with reference to FIG. 9A were subjected to cell killing experiments.

Daudi cells were used as target cells, and the effector cells were FR806-CAR19 T cells and CAR19 T cells. The effector: target ratios were 20:1, 10:1, 5:1, 2.5:1, respectively, the number of target cells was 10000/well, and different numbers of effector cells were set according to different effector: target ratios. 5 duplicate wells were set for each group. In the experimental group, FR806-CAR19 T cells and CAR19 T cells were co-incubated with Daudi cells, and in the control group, T cells infected with Mock virus were incubated with Daudi cells. After 4 hours of incubation, the LDH content in the supernatant was determined by CytoTox96 non-radioactive cytotoxicity kit (Promega), and killing activities were calculated (see the instructions of the CytoTox 96 non-radioactive cytotoxicity kit). Results are shown in FIG. 10A, demonstrating that the cytotoxic activity of FR806-CAR19 T cells was slightly better than that of CAR19 T cells.

CAR19 T cells, CAR19-FR806 T cells and empty plasmid-transfected T cells (Mock) were incubated with Daudi cells for 24 h according to the effector: target ratio=1:1. ELISA was used to detect the secretion level of IFN-γ, IL-2 and TNF-α. Results are shown in FIG. 10B, demonstrating that expression of FR806 has little effects on the level of cytokine release from CAR-T cells.

### Example 9. In Vitro Killing Effects of CH12-MMAF on FR806-CAR19 T Cells

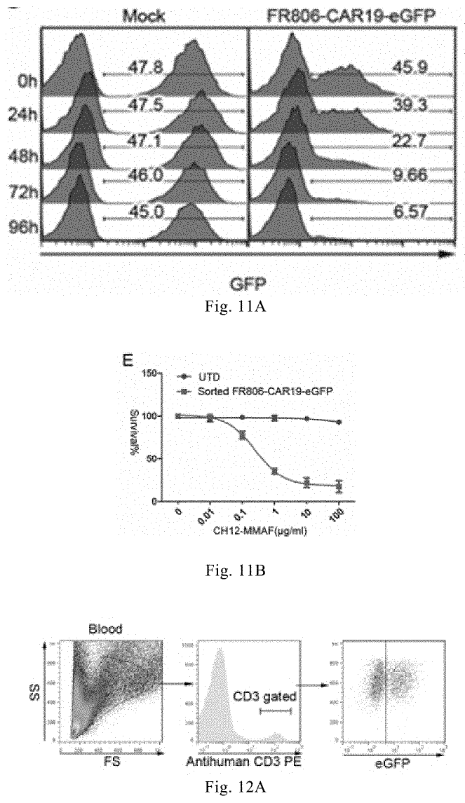

The initial positive rate of FR806-CAR19 T cells and control mock linked according to FIG. 8A was adjusted to 50%, and 10 μg/ml of CH12-MMAF was added, and the positive rate of eGFP was detected by flow cytometry every 24 hours for 96 hours. Results are shown in FIG. 11A, at 24 h, the number of T cells of FR806-CAR19 was decreased, and at 72 hours, the number of T cells of FR806-CAR19 was decreased by about 80%.

FR806-CAR19 T cells were plated in 96-well plates at 3×10⁴ cells per well in 100 ul of medium, 5 replicate wells were set for each drug concentration, and a blank group of medium was also set. Control group: T cells that were not infected with the virus were plated in a 96-well plate with reference to the operation of the experimental group. Six concentrations of CH12-MMAF of 100 μg/ml, 10 μg/ml, 1 μg/ml, 0.1 μg/ml, 0.01 μg/ml and 0 g/ml were added to T cells in the experimental group and the control group, respectively to prepare six gradients (i.e., the aforementioned six concentrations 100 μg/ml, 10 μg/ml, 1 μg/ml, 0.1 μg/ml, 0.01 μg/ml, 0 μg/ml). After 72 h, 10 ul of CCK8 reagent (Dojindo) was added to each well for 3 h at 37° C., and the absorbance was measured at 450 nm by a microplate reader to calculate the cell viability.

The calculation formula is: cell viability (%)=[A (dosing)−A (blank)]/[A (0 dosing)−A (blank)]

The results are shown in FIG. 11B, demonstrating that CH12-MMAF specifically kills FR806-positive CAR T cells without killing Mock cells.

### Example 10: Determination of In Vivo Killing Effects of CH12-MMAF on FR806-CAR19 T Cells

FR806-CAR19 T cells obtained according to FIG. 8A were subjected to the following experiment.

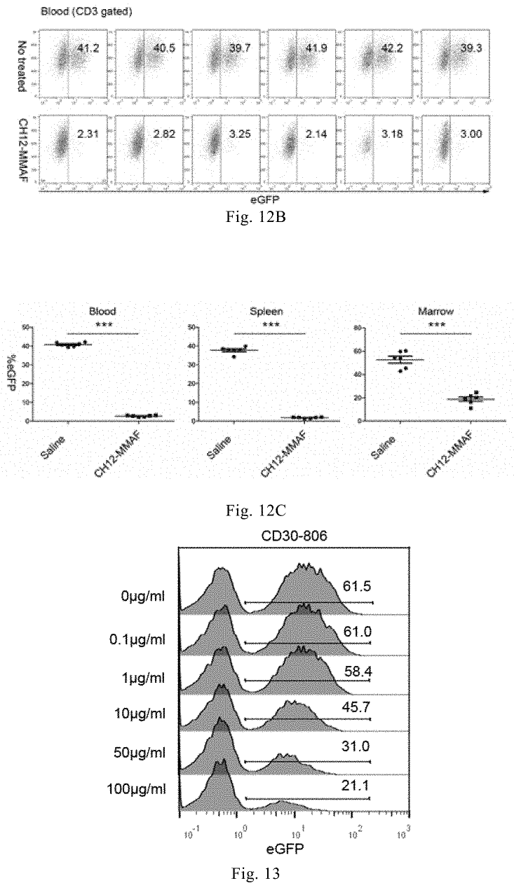

NOD/SCID mice were inoculated with 3×10⁶ Daudi cells, and on day 12, NOD/SCID mice were exposed to cyclophosphamide (100 mg/kg). On day 14, mice were injected with FR806-CAR19 T cells (3×10⁷ cells/animal) via tail vein. On day 15, the experimental group was administered with CH12-MMAF, 0.1 mg/animal, and the control group was given physiological saline. On day 18, the peripheral blood, bone marrow and spleen of the mice were taken, and the red blood cells were lysed by erythrocyte lysate (ebioscience). After washed with PBS, PE-labeled goat anti-human CD3 antibody (1:50, diluted with PBS containing 1% FBS) was added, incubated at 4° C. for 45 minutes, and washed in PBS containing 1% FBS. eGFP positive rate was analyzed by flow cytometry as shown in FIG. 12A.

The results were shown in 11B and 11C. After administration of CH12-MMAF, human CD3⁺/eGFP⁺ cells were reduced by 93% in blood, by 94% in spleen and by 64% in bone marrow; while in the control group, the amounts of human CD3⁺/eGFP⁺ cells detected in blood, spleen and bone marrow were 40.8%, 37.7% and 52.8%, respectively. The results indicated that CH12-MMAF can effectively eliminate FR806-CAR19 T cells in mice.

### Example 11. Expression of eGFP-F2A-CD30806 in T Cells

In this example, eGFP was selected as a fluorescent marker for analysis and eGFP was enhanced green fluorescent protein. F2A was selected as a self-cleaving sequence, which is a core sequence derived from 2A of foot-and-mouth disease virus (or “self-cleaving polypeptide 2A”), has a “self-cleaving” function of 2A and can achieve co-expression of upstream and downstream genes. A partial amino acid sequence of CD30 (SEQ ID NO: 44) and a partial sequence of EGFR (SEQ ID NO: 28) were selected to be expressed as fusion protein CD30806, and the signal peptide of CD30 was selected. The following genetic engineering operations were performed using standard methods known to a skilled person. The nucleotide of eGFP-F2A-CD30806 (SEQ ID NO: 20) was prepared as follows:

SEQ ID NO: 20

Among them, eGFP is shown in bold, F2A is underlined, CD30 SP is shown in bold and underlined, 806 is shown in italics, linker is shown in italics and underlined, and the rest are CD30 receptor transmembrane and intracellular segments.

Atggtgagcaagggcgaggagctgttcaccggggtggtgcccatcctggt
cgagctggacggcgacgtaaacggccacaagttcagcgtgtccggcgagg
gcgagggcgatgccacctacggcaagctgaccctgaagttcatctgcacc
accggcaagctgcccgtgccctggcccaccctcgtgaccaccctgaccta
cggcgtvagtgcttcagccgctaccccgaccacatgaagcagcacgactt
cttcaagtccgccatgcccgaaggctacgtccaggagcgcaccatcttct
tcaaggacgacggcaactacaagacccgcgccgaggtgaagttcgagggc
gacaccctggtgaaccgcatcgagctgaagggcatcgacttcaaggagga
cggcaacatcctggggcacaagctggagtacaactacaacagccacaacg
tctatatcatggccgacaacagaagaacggcatcaaggtgaacttcaaga
tccgccacaacatcgaggacggcagcgtgcagctcgccgaccactaccag
cagaacaccccatcggcgacggccccgtgctgctgcccgacaaccactac
ctgagcacccagtccgccctgagcaaagaccccaacgagaagcgcgatca
catggtcctgctggagttcgtgaccgccgccgggatcactctcggcatgg
acgagctgtacaagtccggagtgaaacagactttgaattttgaccttctg
aagttggcaggagacgttgagtccaaccctgggcccatgcgcgtcctcct
cgccgcgctgggactgctgttcctgggggcgctacgagccgtccgagcct
gtggggccgacagctatgagatggaggaagacggcgtccgcaagtgtaag
aagggtggaggcggttcaggcggaggtggctctggcggtggcggatcgcc
agtgctcttctgggtgatcctggtgttggttgtggtggtcggctccagcg
ccttcctcctgtgccaccggagggcctgcaggaagcgaattcggcagaag
ctccacctgtgctacccggtccagacctcccagcccaagctagagcttgt
ggattccagacccaggaggagctcaacgcagctgaggagtggtgcgtcgg
tgacagaacccgtcgcggaagagcgagggttaatgagccagccactgatg
gagacctgccacagcgtgggggcagcctacctggagagcctgccgctgca
ggatgccagcccggccgggggcccctcgtcccccagggaccttcctgagc
cccgggtgtccacggagcacaccaataacaagattgagaaaatctacatc
atgaaggctgacaccgtgatcgtggggaccgtgaaggctgagctgccgga
gggccggggcctggcggggccagcagagcccgagttggaggaggagctgg
aggcggaccataccccccactaccccgagcaggagacagaaccgcctctg
ggcagctgcagcgatgtcatgctctcagtggaagaggaagggaaagaaga
ccccttgcccacagctgcctctggaaag

The amino acid sequence of eGFP-F2A-CD30806 (SEQ ID NO: 21) is:

Mvskgeelftgvvpilveldgdvnghkfsvsgegegdatygkltlkfict
tgklpvpwptlvttltygvqcfsrypdhmkqhdffksampegyvqertif
fkddgnyktraevkfegdtlvnrielkgidfkedgnilghkleynynshn
vyimadkqkngikvnfkirhniedgsvqladhyqqntpigdgpvllpdnh
ylstqsalskdpnekrdhmvllefvtaagitlgmdelyksgvkqtlnfdl
lklagdvesnpgpmrvllaalgllflgalravracgadsyemeedgvrkc
kkggggsggggsggggspvlfwvilvlvvvvgssafllchrracrkrirq
klhlcypvqtsqpklelvdsrprrsstqlrsgasvtepvaeerglmsqpl
metchsvgaayleslplqdaspaggpssprdlpeprvstehtnnkiekiy
imkadtvivgtvkaelpegrglagpaepeleeeleadhtphypeqetepp
lgscsdvmlsveeegkedplptaasgk

1. Preparation of Nucleotide Sequence of eGFP-F2A-CD30806

1.1 Nucleotide sequences of CD30 signal peptide as shown in SEQ ID NO: 22 and CD30 receptor transmembrane region and intracellular segment as SEQ ID NO: 23 were prepared and obtained according to the experimental procedure in Cell. 1992 Feb. 7; 68(3): 421-7 and the sequence of Genebank accession number NM_001243.4.

SEQ ID NO: 22
Atgcgcgtcctcctcgccgcgctgggactgctgttcctgggggcgctacg
agcc
SEQ ID NO: 23
ccagtgctcttctgggtgatcctggtgttggttgtggtggtcggctccag
cgccttcctcctgtgccaccggagggcctgcaggaagcgaattcggcaga
agctccacctgtgctacccggtccagacctcccagcccaagctagagctt
gtggattccagacccaggaggagctcaacgcagctgaggagtggtgcgtc
ggtgacagaacccgtcgcggaagagcgagggttaatgagccagccactga
tggagacctgccacagcgtgggggcagcctacctggagagcctgccgctg
caggatgccagcccggccgggggcccctcgtcccccagggaccttcctga
gccccgggtgtccacggagcacaccaataacaagattgagaaaatctaca
tcatgaaggctgacaccgtgatcgtggggaccgtgaaggctgagctgccg
gagggccggggcctggcggggccagcagagcccgagttggaggaggagct
ggaggcggaccataccccccactaccccgagcaggagacagaaccgcctc
tgggcagctgcagcgatgtcatgctctcagtggaagaggaagggaaagaa
gaccccttgcccacagctgcctctggaaag

The nucleotide sequence of epidermal growth factor receptor 284-304 epitope (SEQ ID NO: 5) was prepared according to the experimental procedure in Journal of Biological Chemistry, 2004, 279(29), 30375-30384 and the sequence of Genebank Accession No. X00588.1.

SEQ ID NO: 5
Gtccgagcctgtggggccgacagctatgagatggaggaagacggcgtccg
caagtgtaagaag

The nucleotide sequence of the linker (SEQ ID NO: 24) connecting 806 epitope and CD30 transmembrane and intracellular segments was obtained according to the sequence GPC3-Z (SEQ ID NO: 18) in CN application (CN201310164725.X) regarding the nucleic acid encoding GPC-3 chimeric antigen receptor protein and T lymphocytes expressing GPC-3 chimeric antigen receptor protein.

SEQ ID NO: 24
ggtggaggcggttcaggcggaggtggctctggcggtggcggatcg
(a linker in GPC3-Z)

The nucleotide sequence SEQ ID NO: 22, nucleotide sequence SEQ ID NO: 23, nucleotide sequence SEQ ID NO: 24 and nucleotide sequence SEQ ID NO: 5 were sequentially combined and Suzhou Jinweizhi Biotechnology Co., Ltd. was entrusted to carry out the whole genome synthesis, so as to obtain gene fragments of the nucleotide sequence of CD30806 (SEQ ID NO: 25).

SEQ ID NO: 25
Atgcgcgtcctcctcgccgcgctgggactgctgttcctgggggcgctacg
agccgtccgagcctgtggggccgacagctatgagatggaggaagacggcg
tccgcaagtgtaagaagggtggaggcggttcaggcggaggtggctctggc
ggtggcggatcgccagtgctcttctgggtgatcctggtgttggttgtggt
ggtcggctccagcgccttcctcctgtgccaccggagggcctgcaggaagc
gaattcggcagaagctccacctgtgctacccggtccagacctcccagccc
aagctagagcttgtggattccagacccaggaggagctcaacgcagctgag
gagtggtgcgtcggtgacagaacccgtcgcggaagagcgagggttaatga
gccagccactgatggagacctgccacagcgtgggggcagcctacctggag
agcctgccgctgcaggatgccagcccggccgggggcccctcgtcccccag
ggaccttcctgagccccgggtgtccacggagcacaccaataacaagattg
agaaaatctacatcatgaaggctgacaccgtgatcgtggggaccgtgaag
gctgagctgccggagggccggggcctggcggggccagcagagcccgagtt
ggaggaggagctggaggcggaccataccccccactaccccgagcaggaga
cagaaccgcctctgggcagctgcagcgatgtcatgctctcagtggaagag
gaagggaaagaagaccccttgcccacagctgcctctggaaag

1.2. In order to obtain eGFP nucleic acid fragments containing F2A (66 bp) at 3′ end and a small nucleic acid (20 bp) assembled downstream, pWPT-eGFP-F2A-GPC3-BBZ used in CN201310164725.X was used as a template (See SEQ ID NO: 28 in CN201310164725.X for the sequence of the template).

The upstream primer 5′-gcaggggaaagaatagtagaca-3′ (SEQ ID NO: 7) and downstream primer 5′-gcggcgaggaggacgcgcatgggcccagggttggactc-3′ (SEQ ID NO: 26) were used in PCR amplification to obtain eGFP nucleic acid fragments containing F2A (66 bp) at 3′ end and a small nucleic acid (20 bp) assembled downstream.

1.3 Equimolar amount of the eGFP nucleic acid fragments containing F2A (66 bp) at 3′ end and a small nucleic acid (20 bp) assembled downstream obtained in step 1.2 and the CD30806 nucleotide sequence fragments obtained in step 1.1 were linked and subjected to PCR. The DNA polymerase was supplemented, and the upstream primer 5′-gcaggggaaagaatagtagaca-3′ (SEQ ID NO:7) and the downstream primer 5′-ctcgaggtcgacctactttccagaggcagctg-3′ (SEQ ID NO: 27) were added and subjected to PCR for 25 cycles to obtain gene fragments of the nucleotide sequence of eGFP-F2A-CD30806 containing Mul I and Sal I cleavage sites at both ends, the theoretical size of which is 2023 bp, and the amplified product was confirmed by agarose gel electrophoresis to be in agreement with the theoretical size.

2. Construction of eGFP-F2A-CD30806 Lentiviral Vector

The vector system used in the lentiviral plasmid vector used in this example belongs to the third generation of auto-inactivated lentiviral vector system, and the system comprises: packaging plasmid psPAX2 encoding protein Gag/Pol, encoding Rev protein, envelope plasmid PMD2.G encoding VSV-G protein and a recombinant expression vector encoding the target gene eGFP-F2A-FR806 based on empty vector pWPT-eGFP.

In the empty vector pWPT-eGFP, the promoter of elongation factor-1α (elongation factor-1α, EF-1α) regulates the expression of enhanced green fluorescent protein (eGFP), while in the recombinant expression vector encoding the target gene eGFP-F2A-FR806, eGFP was co-expressed with the target gene FR806 by a ribosomal skipping sequence of food and mouth disease virus (FMDV, F2A).

The gene fragments of the nucleotide sequence of eGFP-F2A-CD30806 containing Mul I and Sal I cleavage sites at both ends obtained in example 1.1 were digested by MluI and SalI restriction enzymes, and ligated into pWPT vector which was also double-digested, so as to construct a plasmid pWPT-eGFP-F2A-CD30806 co-expressing eGFP and CD30806 linked by F2A. T cells expressing CD30-806 fusion protein and eGFP were obtained through virus packaging and T cell transfection.

CAR-T cell killing activity experiment: T cells infected with eGFP-CD30806 (abbreviated as CD30-806) were taken, plated at a density of 3×10⁵, different concentrations of CH12-MMAF were added in each well, cells were collected after 72 h, and the proportion of eGFP-positive cells (i.e., CD30-806 positive cells) per well was observed by flow cytometry. The results are shown in FIG. 13. With the increase of the concentration of CH12-MMAF, the proportion of CD30-806 positive cells decreased gradually, indicating that CH12-MMAF exhibits strong killing toxicity against CD30-806 positive cells.

All references mentioned in the present invention are incorporated herein by reference, as if each reference was individually incorporated by reference. In addition, it should be understood that after reading the above teachings of the present invention, those skilled in the art can make various modifications or changes to the present invention, and these equivalent forms also fall within the scope of the appended claims of the present application.

1. An immune effector cell expressing a chimeric antigen receptor on its surface, wherein the immune cell further expresses a fusion protein of formula I 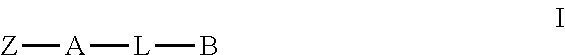
Wherein Z is an optional signal peptide;
A is an antibody binding region;
L is an optional linker moiety; and
B is an endocytic domain.

2. An immune effector cell expressing a chimeric antigen receptor, wherein the immune cell further expresses a fusion protein comprising an antibody binding region and an endocytic domain.

3. The immune effector cell of claim 1 or 2, wherein the antibody binding region is selected from the following antigens or fragments thereof: EGFRvIII, EGFR, CD20, CD22, CD19, BCMA, proBDNF precursor protein, GPC3, CLD18.2, CLD6, mesothelin, PD-L1, PD-1, WT-1, IL13Ra2, Her-2, Her-1, Her-3;
Preferably, the antibody binding region comprises any one of the following amino acid sequences or comprises an amino acid sequence having at least 90%, 91%, 92%, 93%, 94%, 95%, 96%, 97%, 98% or 99% identity with the following amino acid sequence: SEQ ID NO: 28, 29, 33, 34, 35, 36, 37, 38, 39, 40, 41, 42, 43;
More preferably, the antibody binding region comprises an active fragment of any one of the following amino acid sequences: SEQ ID NO: 28, 29, 33, 34, 35, 36, 37, 38, 39, 40, 41, 42, 43.

4. The immune effector cell of claim 3, wherein the antibody binding region specifically binds to an EGFR antibody.

5. The immune effector cell of claim 1 or 2, wherein the endocytic domain is derived from a folate receptor, LDL, CD30, CD33, CD3, EGFR, TFR1; preferably, derived from a folate receptor and CD30; more preferably, the endocytic domain has an amino acid sequence of SEQ ID NO: 32 or 44, or an amino acid sequence having at least 90%, 91%, 92%, 93%, 94%, 95%, 96%, 97%, 98% or 99% identity with SEQ ID NO: 32 or 44, or is an active fragment of an amino acid sequence of SEQ ID NO: 32 or 44.

6. The immune effector cell of claim 1, wherein the signal peptide is a folate receptor signal peptide.

7. The immune effector cell of claim 6, wherein the fusion protein has an amino acid sequence of SEQ ID NO: 10 or comprises an amino acid sequence having at least 90%, 91%, 92%, 93%, 94%, 95%, 96%, 97%, 98% or 99% identity with SEQ ID NO: 10.

8. The immune effector cell of claim 1 or 2, wherein the fusion protein and the chimeric antigen receptor are separately expressed or fusion-expressed on the surface of the immune effector cell, preferably separately expressed.

9. An immune effector cell expressing a chimeric antigen receptor, wherein the cell further expresses an endocytic domain, and the endocytic domain is capable of transferring a substance binding to the endocytic domain into the immune effector cell.

10. The immune effector cell of claim 9, wherein the endocytic domain is derived from a folate receptor, LDL, CD30, CD33, CD3, EGFR, TFR1; preferably, derived from a folate receptor and CD30; more preferably, the endocytic domain having an amino acid sequence of SEQ ID NO:
32 or 44, or an amino acid sequence having at least 90%, 91%, 92%, 93%, 94%, 95%, 96%, 97%, 98% or 99% identity with SEQ ID NO: 32 or 44, or an active fragment of an amino acid sequence of SEQ ID NO: 32 or 44.

11. The immune effector cell of claim 9 or 10, wherein the endocytic domain and the chimeric antigen receptor are separately expressed or fusion-expressed on the surface of the immune effector cell, preferably separately expressed.

12. A fusion protein of Formula I 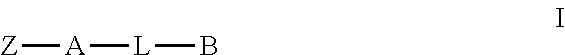
Wherein Z is an optional signal peptide;
A is an antibody binding region;
L is an optional linker moiety; and
B is an endocytic domain.

13. A fusion protein comprising an antibody binding region and an endocytic domain.

14. The fusion protein of claim 12 or 13, wherein the antibody binding region is selected from the following antigens or fragments thereof: EGFRvIII, EGFR, CD20, CD22, CD19, BCMA, proBDNF precursor protein, GPC3, CLD18.2, CLD6, mesothelin, PD-L1, PD-1, WT-1, IL13Ra2, Her-2, Her-1, Her-3;
Preferably, the antibody binding region comprises any one of the following amino acid sequences or comprises an amino acid sequence having at least 90%, 91%, 92%, 93%, 94%, 95%, 96%, 97%, 98% or 99% identity with the following amino acid sequence: SEQ ID NO: 28, 29, 33, 34, 35, 36, 37, 38, 39, 40, 41, 42, 43;
More preferably, the antibody binding region comprises an active fragment of any one of the following amino acid sequences: SEQ ID NO: 28, 29, 33, 34, 35, 36, 37, 38, 39, 40, 41, 42, 43.

15. The fusion protein of claim 14, wherein the antibody binding region specifically binds to an EGFR antibody.

16. The fusion protein of claim 12 or 13, wherein the endocytic domain is derived from a folate receptor, LDL, CD30, CD33, CD3, EGFR, TFR1; preferably derived from a folate receptor and CD30; more preferably, the endocytic domain has an amino acid sequence of SEQ ID NO: 32 or 44, or an amino acid sequence having at least 90%, 91%, 92%, 93%, 94%, 95%, 96%, 97%, 98% or 99% identity with SEQ ID NO: 32 or 44, or is an active fragment of an amino acid sequence of SEQ ID NO: 32 or 44.

17. The fusion protein of claim 12, wherein the signal peptide is a folate receptor signal peptide.

18. The fusion protein of claim 17, wherein the fusion protein has an amino acid sequence of SEQ ID NO: 10 or comprises an amino acid sequence having at least 90%, 91%, 92%, 93%, 94%, 95%, 96%, 97%, 98% or 99% identity with SEQ ID NO: 10.

19. An encoding nucleic acid of the fusion protein of any one of claims 12-18.

20. An expression vector comprising the encoding nucleic acid of claim 19.

21. A host cell, comprising the expression vector of claim 20 or having the encoding nucleic acid of claim 19 integrated into its genome.

22. An immunoconjugate comprising:
A cell-killing functional moiety; and
An antibody that specifically binds to the antibody binding region or endocytic domain in the immune effector cell of any one of claims 1-8, or an antibody that specifically binds to the endocytic domain in an immune effector cell of any one of claims 9-11.

23. Use of the immunoconjugate of claim 22 for specifically killing the immune effector cells of any one of claims 1-11.

24. A kit, comprising the immune effector cell of any one of claims 1-11 or the immunoconjugate of claim 22.

25. A method for specifically eliminating the immune effector cells of any one of claims 1-11, comprising the step of administering the immunoconjugate of claim 22.

26. A method for sorting or enriching the immune effector cells of any one of claims 1-11, comprising the steps of:
Adding a sorting reagent to the system comprising the immune effector cell, wherein the sorting reagent comprises a substance capable of specifically binding to the antibody binding region or endocytic domain in the immune effector cell of any one of claims 1-8, or a substance capable of specifically binding to the endocytic domain in the immune effector cell of any one of claims 1-11; and
A step of separating the substance binding to the immune effector cells from the system.

27. The method of claim 26, wherein the substance capable of specifically binding to the antibody binding region or endocytic domain in the immune effector cell of any one of claims 1-8, or the substance capable of specifically binding to the endocytic domain in the immune effector cell of any one of claims 9-11 is immobilized on a solid phase carrier, thereby separating the substance binding to the immune effector cells from the system.

28. A method for detecting an immune effector cell of any one of claims 1-11, comprising:
Administering a detection reagent that specifically binds to an antibody binding region or endocytic domain in the immune effector cell of any one of claims 1-8 or a detection reagent that specifically binds to the endocytic domain in the immune effector cell of any one of claims 9-11, wherein the detection reagent is linked to a detectable label; and
Detecting a complex formed by the detection reagent and the immune effector cell.


SEQUENCE LISTING (46 sequences)

SEQ ID NO: 1 - DNA, 1623 bp, Artificial Sequence
  Nucleotide sequence of eGFP-F2A-FR806
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtccggagtgaaacagactttgaattttgaccttctgaagttggcaggagacgttgagtccaaccctgggcccatggctcagcggatgacaacacagctgctgctccttctagtgtgggtggctgtagtaggggaggctcagacagtccgagcctgtggggccgacagctatgagatggaggaagacggcgtccgcaagtgtaagaagaggattgcatgggccaggactgagcttctcaatgtctgcatgaacgccaagcaccacaaggaaaagccaggccccgaggacaagttgcatgagcagtgtcgaccctggaggaagaatgcctgctgttctaccaacaccagccaggaagcccataaggatgtttcctacctatatagattcaactggaaccactgtggagagatggcacctgcctgcaaacggcatttcatccaggacacctgcctctacgagtgctcccccaacttggggccctggatccagcaggtggatcagagctggcgcaaagagcgggtactgaacgtgcccctgtgcaaagaggactgtgagcaatggtgggaagattgtcgcacctcctacacctgcaagagcaactggcacaagggctggaactggacttcagggtttaacaagtgcgcagtgggagctgcctgccaacctttccatttctacttccccacacccactgttctgtgcaatgaaatctggactcactcctacaaggtcagcaactacagccgagggagtggccgctgcatccagatgtggttcgacccagcccagggcaaccccaatgaggaggtggcgaggttctatgctgcagccatgagtggggctgggccctgggcagcctggcctttcctgcttagcctggccctaatgctgctgtggctgctcagc

SEQ ID NO: 2 - protein, 541 aa, Artificial Sequence
  Amino acid sequence of eGFP-F2A-FR806
MVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYKSGVKQTLNFDLLKLAGDVESNPGPMAQRMTTQLLLLLVWVAVVGEAQTVRACGADSYEMEEDGVRKCKKRIAWARTELLNVCMNAKHHKEKPGPEDKLHEQCRPWRKNACCSTNTSQEAHKDVSYLYRFNWNHCGEMAPACKRHFIQDTCLYECSPNLGPWIQQVDQSWRKERVLNVPLCKEDCEQWWEDCRTSYTCKSNWHKGWNWTSGFNKCAVGAACQPFHFYFPTPTVLCNEIWTHSYKVSNYSRGSGRCIQMWFDPAQGNPNEEVARFYAAAMSGAGPWAAWPFLLSLALMLLWLLS

SEQ ID NO: 3 - DNA, 72 bp, Artificial Sequence
  Nucleotide sequence of FOLR1 signal peptide
atggctcagcggatgacaacacagctgctgctccttctagtgtgggtggctgtagtaggggaggctcagaca

SEQ ID NO: 4 - DNA, 699 bp, Artificial Sequence
  Nucleotide sequence of remaining part of FOLR1
aggattgcatgggccaggactgagcttctcaatgtctgcatgaacgccaagcaccacaaggaaaagccaggccccgaggacaagttgcatgagcagtgtcgaccctggaggaagaatgcctgctgttctaccaacaccagccaggaagcccataaggatgtttcctacctatatagattcaactggaaccactgtggagagatggcacctgcctgcaaacggcatttcatccaggacacctgcctctacgagtgctcccccaacttggggccctggatccagcaggtggatcagagctggcgcaaagagcgggtactgaacgtgcccctgtgcaaagaggactgtgagcaatggtgggaagattgtcgcacctcctacacctgcaagagcaactggcacaagggctggaactggacttcagggtttaacaagtgcgcagtgggagctgcctgccaacctttccatttctacttccccacacccactgttctgtgcaatgaaatctggactcactcctacaaggtcagcaactacagccgagggagtggccgctgcatccagatgtggttcgacccagcccagggcaaccccaatgaggaggtggcgaggttctatgctgcagccatgagtggggctgggccctgggcagcctggcctttcctgcttagcctggccctaatgctgctgtggctgctcagc

SEQ ID NO: 5 - DNA, 63 bp, Artificial Sequence
  Nucleotide sequence of 284-304 epitope of EGFR
gtccgagcctgtggggccgacagctatgagatggaggaagacggcgtccgcaagtgtaagaag

SEQ ID NO: 6 - DNA, 834 bp, Artificial Sequence
  Nucleotide sequence of FR806
atggctcagcggatgacaacacagctgctgctccttctagtgtgggtggctgtagtaggggaggctcagacagtccgagcctgtggggccgacagctatgagatggaggaagacggcgtccgcaagtgtaagaagaggattgcatgggccaggactgagcttctcaatgtctgcatgaacgccaagcaccacaaggaaaagccaggccccgaggacaagttgcatgagcagtgtcgaccctggaggaagaatgcctgctgttctaccaacaccagccaggaagcccataaggatgtttcctacctatatagattcaactggaaccactgtggagagatggcacctgcctgcaaacggcatttcatccaggacacctgcctctacgagtgctcccccaacttggggccctggatccagcaggtggatcagagctggcgcaaagagcgggtactgaacgtgcccctgtgcaaagaggactgtgagcaatggtgggaagattgtcgcacctcctacacctgcaagagcaactggcacaagggctggaactggacttcagggtttaacaagtgcgcagtgggagctgcctgccaacctttccatttctacttccccacacccactgttctgtgcaatgaaatctggactcactcctacaaggtcagcaactacagccgagggagtggccgctgcatccagatgtggttcgacccagcccagggcaaccccaatgaggaggtggcgaggttctatgctgcagccatgagtggggctgggccctgggcagcctggcctttcctgcttagcctggccctaatgctgctgtggctgctcagc

SEQ ID NO: 7 - DNA, 22 bp, Artificial Sequence
  Primer
gcaggggaaagaatagtagaca

SEQ ID NO: 8 - DNA, 38 bp, Artificial Sequence
  Primer
gttgtcatccgctgagccatgggcccagggttggactc

SEQ ID NO: 9 - DNA, 31 bp, Artificial Sequence
  Primer
ctcgaggtcgacctagctgagcagccacagc

SEQ ID NO: 10 - protein, 278 aa, Artificial Sequence
  Sequence of FR806 fusion protein (comprising signal peptide)
MAQRMTTQLLLLLVWVAVVGEAQTVRACGADSYEMEEDGVRKCKKRIAWARTELLNVCMNAKHHKEKPGPEDKLHEQCRPWRKNACCSTNTSQEAHKDVSYLYRFNWNHCGEMAPACKRHFIQDTCLYECSPNLGPWIQQVDQSWRKERVLNVPLCKEDCEQWWEDCRTSYTCKSNWHKGWNWTSGFNKCAVGAACQPFHFYFPTPTVLCNEIWTHSYKVSNYSRGSGRCIQMWFDPAQGNPNEEVARFYAAAMSGAGPWAAWPFLLSLALMLLWLLS

SEQ ID NO: 11 - DNA, 750 bp, Artificial Sequence
  Nucleotide fragment of single-chain antibody of CD19
gatatccagctgacccagtctccagcttctttggctgtgtctctagggcagagggccaccatctcctgcaaggccagccaaagtgttgattatgatggtgatagttatttgaactggtaccaacagattccaggacagccacccaaactcctcatctatgatgcatccaatctagtttctgggatcccacccaggtttagtggcagtgggtctgggacagacttcaccctcaacatccatcctgtggagaaggtggatgctgcaacctatcactgtcagcaaagtactgaggatccgtggacgttcggtggagggaccaagctcgagatcaaaggtggtggtggttctggcggcggcggctccggtggtggtggttctcaggtgcagctgcagcagtctggggctgagctggtgaggcctgggtcctcagtgaagatttcctgcaaggcttctggctatgcattcagtagctactggatgaactgggtgaagcagaggcctggacagggtcttgagtggattggacagatttggcctggagatggtgatactaactacaatggaaagttcaagggtaaagccactctgactgcagacgaatcctccagcacagcctacatgcaactcagcagcctagcatctgaggactctgcggtctatttctgtgcaagacgggagactacgacggtaggccgttattactatgctatggactactggggccaagggaccacggtcaccgtctcctcc

SEQ ID NO: 12 - DNA, 1485 bp, Artificial Sequence
  Nucleotide sequence of alpha CD19CAR
atggccttaccagtgaccgccttgctcctgccgctggccttgctgctccacgccgccaggccggatatccagctgacccagtctccagcttctttggctgtgtctctagggcagagggccaccatctcctgcaaggccagccaaagtgttgattatgatggtgatagttatttgaactggtaccaacagattccaggacagccacccaaactcctcatctatgatgcatccaatctagtttctgggatcccacccaggtttagtggcagtgggtctgggacagacttcaccctcaacatccatcctgtggagaaggtggatgctgcaacctatcactgtcagcaaagtactgaggatccgtggacgttcggtggagggaccaagctcgagatcaaaggtggtggtggttctggcggcggcggctccggtggtggtggttctcaggtgcagctgcagcagtctggggctgagctggtgaggcctgggtcctcagtgaagatttcctgcaaggcttctggctatgcattcagtagctactggatgaactgggtgaagcagaggcctggacagggtcttgagtggattggacagatttggcctggagatggtgatactaactacaatggaaagttcaagggtaaagccactctgactgcagacgaatcctccagcacagcctacatgcaactcagcagcctagcatctgaggactctgcggtctatttctgtgcaagacgggagactacgacggtaggccgttattactatgctatggactactggggccaagggaccacggtcaccgtctcctccaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 13 - DNA, 58 bp, Artificial Sequence
  Primer
ccttctgaagttggcaggagacgttgagtccaaccctgggcccatggccttaccagtg

SEQ ID NO: 14 - DNA, 57 bp, Artificial Sequence
  Primer
tcctgccaacttcagaaggtcaaaattcaaagtctgtttcacgcgagggggcagggc

SEQ ID NO: 15 - DNA, 3168 bp, Artificial Sequence
  Nucleotide sequence of FR806-F2A-CAR19-F2A-eGFP
atggctcagcggatgacaacacagctgctgctccttctagtgtgggtggctgtagtaggggaggctcagacagtccgagcctgtggggccgacagctatgagatggaggaagacggcgtccgcaagtgtaagaagaggattgcatgggccaggactgagcttctcaatgtctgcatgaacgccaagcaccacaaggaaaagccaggccccgaggacaagttgcatgagcagtgtcgaccctggaggaagaatgcctgctgttctaccaacaccagccaggaagcccataaggatgtttcctacctatatagattcaactggaaccactgtggagagatggcacctgcctgcaaacggcatttcatccaggacacctgcctctacgagtgctcccccaacttggggccctggatccagcaggtggatcagagctggcgcaaagagcgggtactgaacgtgcccctgtgcaaagaggactgtgagcaatggtgggaagattgtcgcacctcctacacctgcaagagcaactggcacaagggctggaactggacttcagggtttaacaagtgcgcagtgggagctgcctgccaacctttccatttctacttccccacacccactgttctgtgcaatgaaatctggactcactcctacaaggtcagcaactacagccgagggagtggccgctgcatccagatgtggttcgacccagcccagggcaaccccaatgaggaggtggcgaggttctatgctgcagccatgagtggggctgggccctgggcagcctggcctttcctgcttagcctggccctaatgctgctgtggctgctcagcgtgaaacagactttgaattttgaccttctgaagttggcaggagacgttgagtccaaccctgggcccatggccttaccagtgaccgccttgctcctgccgctggccttgctgctccacgccgccaggccggatatccagctgacccagtctccagcttctttggctgtgtctctagggcagagggccaccatctcctgcaaggccagccaaagtgttgattatgatggtgatagttatttgaactggtaccaacagattccaggacagccacccaaactcctcatctatgatgcatccaatctagtttctgggatcccacccaggtttagtggcagtgggtctgggacagacttcaccctcaacatccatcctgtggagaaggtggatgctgcaacctatcactgtcagcaaagtactgaggatccgtggacgttcggtggagggaccaagctcgagatcaaaggtggtggtggttctggcggcggcggctccggtggtggtggttctcaggtgcagctgcagcagtctggggctgagctggtgaggcctgggtcctcagtgaagatttcctgcaaggcttctggctatgcattcagtagctactggatgaactgggtgaagcagaggcctggacagggtcttgagtggattggacagatttggcctggagatggtgatactaactacaatggaaagttcaagggtaaagccactctgactgcagacgaatcctccagcacagcctacatgcaactcagcagcctagcatctgaggactctgcggtctatttctgtgcaagacgggagactacgacggtaggccgttattactatgctatggactactggggccaagggaccacggtcaccgtctcctccaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcgtgaaacagactttgaattttgaccttctgaagttggcaggagacgttgagtccaaccctgggcccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaag

SEQ ID NO: 16 - DNA, 46 bp, Artificial Sequence
  Primer
cttacgcgtcctagcgctaccggtcgccaccatggctcagcggatg

SEQ ID NO: 17 - DNA, 58 bp, Artificial Sequence
  Primer
gtctcctgccaacttcagaaggtcaaaattcaaagtctgtttcacgctgagcagccac

SEQ ID NO: 18 - DNA, 59 bp, Artificial Sequence
  Primer
accttctgaagttggcaggagacgttgagtccaaccctgggcccatggtgagcaagggc

SEQ ID NO: 19 - DNA, 28 bp, Artificial Sequence
  Primer
ctcgaggtcgacctacttgtacagctcg

SEQ ID NO: 20 - DNA, 1581 bp, Artificial Sequence
  Nucleotide sequence of eGFP-F2A-CD30806
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtccggagtgaaacagactttgaattttgaccttctgaagttggcaggagacgttgagtccaaccctgggcccatgcgcgtcctcctcgccgcgctgggactgctgttcctgggggcgctacgagccgtccgagcctgtggggccgacagctatgagatggaggaagacggcgtccgcaagtgtaagaagggtggaggcggttcaggcggaggtggctctggcggtggcggatcgccagtgctcttctgggtgatcctggtgttggttgtggtggtcggctccagcgccttcctcctgtgccaccggagggcctgcaggaagcgaattcggcagaagctccacctgtgctacccggtccagacctcccagcccaagctagagcttgtggattccagacccaggaggagctcaacgcagctgaggagtggtgcgtcggtgacagaacccgtcgcggaagagcgagggttaatgagccagccactgatggagacctgccacagcgtgggggcagcctacctggagagcctgccgctgcaggatgccagcccggccgggggcccctcgtcccccagggaccttcctgagccccgggtgtccacggagcacaccaataacaagattgagaaaatctacatcatgaaggctgacaccgtgatcgtggggaccgtgaaggctgagctgccggagggccggggcctggcggggccagcagagcccgagttggaggaggagctggaggcggaccataccccccactaccccgagcaggagacagaaccgcctctgggcagctgcagcgatgtcatgctctcagtggaagaggaagggaaagaagaccccttgcccacagctgcctctggaaag

SEQ ID NO: 21 - protein, 527 aa, Artificial Sequence
  Amino acid sequence of eGFP-F2A-CD30806
MVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYKSGVKQTLNFDLLKLAGDVESNPGPMRVLLAALGLLFLGALRAVRACGADSYEMEEDGVRKCKKGGGGSGGGGSGGGGSPVLFWVILVLVVVVGSSAFLLCHRRACRKRIRQKLHLCYPVQTSQPKLELVDSRPRRSSTQLRSGASVTEPVAEERGLMSQPLMETCHSVGAAYLESLPLQDASPAGGPSSPRDLPEPRVSTEHTNNKIEKIYIMKADTVIVGTVKAELPEGRGLAGPAEPELEEELEADHTPHYPEQETEPPLGSCSDVMLSVEEEGKEDPLPTAASGK

SEQ ID NO: 22 - DNA, 54 bp, Artificial Sequence
  Nucleotide sequence of CD30 signal peptide
atgcgcgtcctcctcgccgcgctgggactgctgttcctgggggcgctacgagcc

SEQ ID NO: 23 - DNA, 630 bp, Artificial Sequence
  Nucleotide sequence of transmembrane and intracellular segments of CD30 receptor
ccagtgctcttctgggtgatcctggtgttggttgtggtggtcggctccagcgccttcctcctgtgccaccggagggcctgcaggaagcgaattcggcagaagctccacctgtgctacccggtccagacctcccagcccaagctagagcttgtggattccagacccaggaggagctcaacgcagctgaggagtggtgcgtcggtgacagaacccgtcgcggaagagcgagggttaatgagccagccactgatggagacctgccacagcgtgggggcagcctacctggagagcctgccgctgcaggatgccagcccggccgggggcccctcgtcccccagggaccttcctgagccccgggtgtccacggagcacaccaataacaagattgagaaaatctacatcatgaaggctgacaccgtgatcgtggggaccgtgaaggctgagctgccggagggccggggcctggcggggccagcagagcccgagttggaggaggagctggaggcggaccataccccccactaccccgagcaggagacagaaccgcctctgggcagctgcagcgatgtcatgctctcagtggaagaggaagggaaagaagaccccttgcccacagctgcctctggaaag

SEQ ID NO: 24 - DNA, 45 bp, Artificial Sequence
  A linker in GPC3-Z
ggtggaggcggttcaggcggaggtggctctggcggtggcggatcg

SEQ ID NO: 25 - DNA, 792 bp, Artificial Sequence
  Nucleotide sequence of CD30806
atgcgcgtcctcctcgccgcgctgggactgctgttcctgggggcgctacgagccgtccgagcctgtggggccgacagctatgagatggaggaagacggcgtccgcaagtgtaagaagggtggaggcggttcaggcggaggtggctctggcggtggcggatcgccagtgctcttctgggtgatcctggtgttggttgtggtggtcggctccagcgccttcctcctgtgccaccggagggcctgcaggaagcgaattcggcagaagctccacctgtgctacccggtccagacctcccagcccaagctagagcttgtggattccagacccaggaggagctcaacgcagctgaggagtggtgcgtcggtgacagaacccgtcgcggaagagcgagggttaatgagccagccactgatggagacctgccacagcgtgggggcagcctacctggagagcctgccgctgcaggatgccagcccggccgggggcccctcgtcccccagggaccttcctgagccccgggtgtccacggagcacaccaataacaagattgagaaaatctacatcatgaaggctgacaccgtgatcgtggggaccgtgaaggctgagctgccggagggccggggcctggcggggccagcagagcccgagttggaggaggagctggaggcggaccataccccccactaccccgagcaggagacagaaccgcctctgggcagctgcagcgatgtcatgctctcagtggaagaggaagggaaagaagaccccttgcccacagctgcctctggaaag

SEQ ID NO: 26 - DNA, 38 bp, Artificial Sequence
  Primer
gcggcgaggaggacgcgcatgggcccagggttggactc

SEQ ID NO: 27 - DNA, 32 bp, Artificial Sequence
  Primer
ctcgaggtcgacctactttccagaggcagctg

SEQ ID NO: 28 - protein, 21 aa, Artificial Sequence
  EGFR284-304 epitope
VRACGADSYEMEEDGVRKCKK

SEQ ID NO: 29 - protein, 16 aa, Artificial Sequence
  EGFR287-302 epitope
CGADSYEMEEDGVRKC

SEQ ID NO: 30 - protein, 22 aa, Artificial Sequence
  Self-cleaving sequence
VKQTLNFDLLKLAGDVESNPGP

SEQ ID NO: 31 - protein, 254 aa, Artificial Sequence
  Amino acid sequence only comprising 806 and other part of folate receptor but not comprising signal peptide
VRACGADSYEMEEDGVRKCKKRIAWARTELLNVCMNAKHHKEKPGPEDKLHEQCRPWRKNACCSTNTSQEAHKDVSYLYRFNWNHCGEMAPACKRHFIQDTCLYECSPNLGPWIQQVDQSWRKERVLNVPLCKEDCEQWWEDCRTSYTCKSNWHKGWNWTSGFNKCAVGAACQPFHFYFPTPTVLCNEIWTHSYKVSNYSRGSGRCIQMWFDPAQGNPNEEVARFYAAAMSGAGPWAAWPFLLSLALMLLWLLS

SEQ ID NO: 32 - protein, 233 aa, Artificial Sequence
  Amino acid sequence of other part of folate receptor
RIAWARTELLNVCMNAKHHKEKPGPEDKLHEQCRPWRKNACCSTNTSQEAHKDVSYLYRFNWNHCGEMAPACKRHFIQDTCLYECSPNLGPWIQQVDQSWRKERVLNVPLCKEDCEQWWEDCRTSYTCKSNWHKGWNWTSGFNKCAVGAACQPFHFYFPTPTVLCNEIWTHSYKVSNYSRGSGRCIQMWFDPAQGNPNEEVARFYAAAMSGAGPWAAWPFLLSLALMLLWLLS

SEQ ID NO: 33 - protein, 6 aa, Artificial Sequence
  GPC3-546-551 epitope
DNEIST

SEQ ID NO: 34 - protein, 57 aa, Artificial Sequence
  GPC3-524-580 epitope
AELAYDLDVDDAPGNSQQATPKDNEISTFHNLGNVHSPLKLLTSMAISVVCFFFLVH

SEQ ID NO: 35 - protein, 40 aa, Artificial Sequence
  GPC3-524-580 epitope
AELAYDLDVDDAPGNSQQATPKDNEISTFHNLGNVHSPLK

SEQ ID NO: 36 - protein, 10 aa, Artificial Sequence
  GPC3-544-553 epitope
PKDNEISTFH

SEQ ID NO: 37 - protein, 55 aa, Artificial Sequence
  18A2-loop1 epitope
MDQWSTQDLYNNPVTAVFNYQGLWRSCVRESSGFTECRGYFTLLGLPAMLQAVRA

SEQ ID NO: 38 - protein, 40 aa, Artificial Sequence
  18A2-loop2 epitope
ANMLVTNFWMSTANMYTGMGGMVQTVQTRYTFGAALFVGW

SEQ ID NO: 39 - protein, 24 aa, Artificial Sequence
  18A2-loopD3 epitope
FALKCIRIGSMEDSAKANMTLTSG

SEQ ID NO: 40 - protein, 54 aa, Artificial Sequence
  Extracellular segment of BCMA
MLQMAGQCSQNEYFDSLLHACIPCQLRCSSNTPPLTCQRYCNASVTNSVKGTNA

SEQ ID NO: 41 - protein, 272 aa, Artificial Sequence
  Extracellular segment of CD19
PEEPLVVKVEEGDNAVLQCLKGTSDGPTQQLTWSRESPLKPFLKLSLGLPGLGIHMRPLAIWLFIFNVSQQMGGFYLCQPGPPSEKAWQPGWTVNVEGSGELFRWNVSDLGGLGCGLKNRSSEGPSSPSGKLMSPKLYVWAKDRPEIWEGEPPCLPPRDSLNQSLSQDLTMAPGSTLWLSCGVPPDSVSRGPLSWTHVHPKGPKSLLSLELKDDRPARDMWVMETGLLLPRATAQDAGKYYCHRGNLTMSFHLEITARPVLWHWLLRTGGWK

SEQ ID NO: 42 - protein, 297 aa, Artificial Sequence
  Full-length CD20
MTTPRNSVNGTFPAEPMKGPIAMQSGPKPLFRRMSSLVGPTQSFFMRESKTLGAVQIMNGLFHIALGGLLMIPAGIYAPICVTVWYPLWGGIMYIISGSLLAATEKNSRKCLVKGKMIMNSLSLFAAISGMILSIMDILNIKISHFLKMESLNFIRAHTPYINIYNCEPANPSEKNSPSTQYCYSIQSLFLGILSVMLIFAFFQELVIAGIVENEWKRTCSRPKSNIVLLSAEEKKEQTIEIKEEVVGLTETSSQPKNEEDIEIIPIQEEEEEETETNFPEPPQDQESSPIENDSSP

SEQ ID NO: 43 - protein, 668 aa, Artificial Sequence
  Extracellular segment of CD22
DSSKWVFEHPETLYAWEGACVWIPCTYRALDGDLESFILFHNPEYNKNTSKFDGTRLYESTKDGKVPSEQKRVQFLGDKNKNCTLSIHPVHLNDSGQLGLRMESKTEKWMERIHLNVSERPFPPHIQLPPEIQESQEVTLTCLLNFSCYGYPIQLQWLLEGVPMRQAAVTSTSLTIKSVFTRSELKFSPQWSHHGKIVTCQLQDADGKFLSNDTVQLNVKHTPKLEIKVTPSDAIVREGDSVTMTCEVSSSNPEYTTVSWLKDGTSLKKQNTFTLNLREVTKDQSGKYCCQVSNDVGPGRSEEVFLQVQYAPEPSTVQILHSPAVEGSQVEFLCMSLANPLPTNYTWYHNGKEMQGRTEEKVHIPKILPWHAGTYSCVAENILGTGQRGPGAELDVQYPPKKVTTVIQNPMPIREGDTVTLSCNYNSSNPSVTRYEWKPHGAWEEPSLGVLKIQNVGWDNTTIACAACNSWCSWASPVALNVQYAPRDVRVRKIKPLSEIHSGNSVSLQCDFSSSHPKEVQFFWEKNGRLLGKESQLNFDSISPEDAGSYSCWVNNSIGQTASKAWTLEVLYAPRRLRVSMSPGDQVMEGKSATLTCESDANPPVSHYTWFDWNNQSLPYHSQKLRLEPVKVQHSGAYWCQGTNSVGKGRSPLSTLTVYYSPETIGRR

SEQ ID NO: 44 - protein, 210 aa, Artificial Sequence
  Transmembrane and intracellular domains of CD30
PVLFWVILVLVVVVGSSAFLLCHRRACRKRIRQKLHLCYPVQTSQPKLELVDSRPRRSSTQLRSGASVTEPVAEERGLMSQPLMETCHSVGAAYLESLPLQDASPAGGPSSPRDLPEPRVSTEHTNNKIEKIYIMKADTVIVGTVKAELPEGRGLAGPAEPELEEELEADHTPHYPEQETEPPLGSCSDVMLSVEEEGKEDPLPTAASGK

SEQ ID NO: 45 - protein, 464 aa, Artificial Sequence
  Heavy chain of CH12
MRVLILLWLFTAFPGFLSDVQLQESGPGLVKPSQSLSLTCTVTAYSVTSDYAWNWIRQFPGNKLEWMGYISYSGTTRYNPSLKSRISITRDTSKNQFFLQLNSMTAEDTATYYCSRQGRGFPYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 46 - protein, 236 aa, Artificial Sequence
  Light chain of CH12
MDMMVLAQFLAFLLLWFPGARCDILMTQSPSSMSVSLGDTVSITCHASQDINSNIGWLQQKPGKSFKGLIYHGTNLEDGVPSRFSGSGSGADYSLTISSLESEDFADYYCVQYAQFPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC